(19) United States
(12) Patent Application Publication
(10) Pub. No.: US 2023/0212219 A1
(43) Pub. Date: Jul. 6, 2023

(54) PROCESS FOR ISOLATING SOLUBLE FUNCTIONAL PROTEINS FROM PLANT MATERIAL

(71) Applicant: Coöperatie Koninklijke Cosun U.A., Breda (NL)

(72) Inventors: André Banier de Haan, Dinteloord (NL); Marlies Elisabeth Johanna Geerts, Dinteloord (NL); Paulus Statius Reinier Kosters, Dinteloord (NL)

(73) Assignee: Coöperatie Koninklijke Cosun U.A., Breda (NL)

(21) Appl. No.: 17/998,931

(22) PCT Filed: May 17, 2021
(86) PCT No.: PCT/NL2021/050315

§ 371 Date: Nov. 16, 2022

(30) Foreign Application Priority Data
May 18, 2020 (NL) 2025601

(51) Int. Cl. C07K 1/36; A23J 1/00; B01D 21/01; B01D 21/26; B04B 1/08; B04B 11/02; B01D 61/14; B01D 15/08; B01D 25/12; C07K 14/415

(52) U.S. Cl. CPC C07K 1/36; A23J 1/007; B01D 21/01; B01D 21/262; B04B 1/08; B04B 11/02; B01D 61/147; B01D 61/145; B01D 61/149; B01D 15/08; B01D 25/12; C07K 14/415; B01D 2315/16

(57) ABSTRACT

A process for obtaining soluble functional proteins from plant material includes the steps of: mechanically disrupting the cells of the plant material to obtain a mush stream; subjecting the mush stream to a coarse physical separation step, resulting in a permeate and a retentate; subjecting the permeate P_{b} to mild treatment, resulting in a treated permeate; subjecting the treated permeate to serial centrifugation steps; subjecting centrate to a microfiltration step resulting in a permeate and a retentate; subjecting the permeate to an ultrafiltration step resulting in a permeate and a retentate; subjecting the retentate to hydrophobic column adsorption to provide a column permeate and a retentate; and drying the column permeate to provide a soluble functional protein isolate.

### FIELD OF THE INVENTION

The present invention relates to a process for obtaining soluble functional proteins from plant material, in particular from green leaves.

### BACKGROUND OF THE INVENTION

Proteins from plant material could potentially form a major protein source for food applications. Leaves from several crops, including leaves that are by-products from agricultural crops, may be suitable sources, depending on their protein content, regional availability, social needs and current uses.

The amount of protein in green leaves typically varies between 1.2 and 8.2 wt. % (by total wet weight of the leaves) depending on the characteristics of the plant and its growing conditions (van de Velde et al., New Food, 2011, 14, pp 10-13).

RuBisCo (ribulose-1,5-bisphosphate carboxylase/oxygenase) is the most abundant protein in green leaves, comprising up to 50% of the soluble proteins in the leaf. The amino acid composition of RuBisCo is favorable for human consumption, since almost all the essential amino acids (e.g. isoleucine, leucine, lysine, methionine, phenylalanine, threonine, tryptophan and valine) are present in relatively high enough amounts. Even more interesting is that RuBisCo is water soluble, has shown to be a good gelling and foaming agent, and is readily digestible and non-allergic.

Accordingly, isolating soluble functional proteins, including RuBisCo, from plant material, such as green leaves, has been the subject of numerous studies.

The basic principles of soluble functional protein isolation from green biomass are described in A.-L Nynäs, (2018), White proteins from green leaves in food applications, A literature study, Alnarp: Sveriges lantbruksuniversitet (Introductory paper at the Faculty of Landscape Architecture, Horticulture and Crop Production Science, 2018:1). The process starts with extracting a green juice by pressing of the green biomass. The chlorophyll-related proteins, also called ‘green proteins’, which are unwanted in terms of taste and smell, are subsequently precipitated from the green juice, resulting in a brown juice. This brown juice comprises the soluble functional proteins, also referred to as the ‘white protein fraction’. The white proteins are then purified and concentrated from this brown juice to obtain a soluble functional protein isolate.

Plant materials not only contain various proteins, but also other compounds, some of which are, even in very low concentrations, highly undesirable in protein isolates in terms of their contribution to colour, smell or taste of the protein isolate. Other compounds may negatively affect the yield of the protein isolate by interfering with the protein during the isolation process.

The most important classes of compounds challenging the purification of soluble functional proteins from plant material are polyphenols, pigments such as chlorophyll and carotenoids, oxidized lipids and proteases. Polyphenols bind covalently to the proteins and thus impair the nutritional value of the proteins, change their functional properties and may reduce the yield of the protein isolation process. Pigments, which are abundantly present in plants, influence heavily the quality of the protein isolate, result in an undesired green colour and in an unwanted grassy smell and taste. The protein-bound oxidized phospholipids may produce during or after the isolation of a plant protein off-odours or off-flavours. The released proteases can have adverse effects on the stability of the proteins and lead to degradation of the desired proteins.

In order to extract soluble functional protein from a plant material, the plant material is usually treated with several chemical agents, such as strong acids and/or strong alkalis, and is typically further processed under high temperatures, pressures and/or external forces. Hence, typically, a large amount of chemicals is used and complex equipment is applied to obtain soluble functional protein from a plant material. This is of great influence on the economics of the process of isolating soluble functional protein from plant material on an industrial scale. Moreover, these chemical agents and harsh process conditions may result in a reduction or even in the loss of the functionality of soluble plant proteins due to denaturation. Denaturation of, for example, RuBisCo decreases its solubility and increases the binding of polyphenols thereto. Hence, in order to retain protein functionality, the extraction and purification of the protein should be as mild as possible, i.e the use of denaturing agents, high temperature and strong acids and alkalis should be avoided.

Another problem related to isolating soluble functional protein from plant material is loss of a significant amount of soluble functional protein as a result of the isolation process itself. About 80% of the leaf proteins are located inside the chloroplast (A. Tamayo Tenorio et al., Food Chemistry, 2016, 203, pp 402-408), wherein a sophisticated membrane system (i.e. thylakoids) contains several membrane protein complexes. The association of RuBisCo with the thylakoid membranes often results in the loss of the majority of RuBisCo during the isolation process. This association is influenced by different kinds of salts, the concentration of these salts, and the pH value of the extraction buffer used.

Knuckles et al., J. Agric. Food Chem., 1975, 23, pp 209-212, disclose a lab-scale process for the isolation of proteins from alfalfa by removing the green chloroplastic proteins from fresh alfalfa juice, followed by ultrafiltration and diafiltration. The proteins are crystallized from the resulting protein concentrate.

WO82/04066A1 and U.S. Pat. No. 4,268,632 disclose a process for isolating proteins from plant leaves, in particular tobacco leaves. After the leaves are ground into a pulp, the supernatant part of the pulp is stored under a temperature at or below room temperature to obtain RuBisCo in crystalline form.

WO2011/078671A1 discloses a process for the isolation of RuBisCo from plant material comprising the following steps:
a) lysing the plant material to release RuBisCo and chlorophyll from the plant cells;
b) separating the lysed plant material into a liquid juice comprising the RuBisCo and a solid phase;
c) removal of the chlorophyll from the liquid juice through addition of activated carbon; and
d) separating the liquid juice and the chlorophyll-loaded activated carbon.

The total amount of isolated soluble functional protein obtained, by this process by weight of the soluble functional protein in the starting plant material, was 8.5% with a purity of 82.6% for carrot leaves and 8.81% with a purity of 92.79% for spinach leaves.

WO2014/104880A1 discloses a method for isolating soluble plant protein from plant material, such as sugar beet leaves, said method comprising the following steps:
a) mechanically disrupting the plant cells of said plant material to obtain a plant juice, wherein before, during, or after the step of disrupting the plant cells an extraction composition comprising at least one of a reducing agent and a divalent ion source is added to said plant material;
b) treating the plant juice to cause aggregation of chloroplast membranes;
c) separating said aggregated chloroplast membranes from the soluble plant protein in said treated plant juice by precipitation and/or microfiltration to provide a plant juice supernatant or plant juice permeate comprising the soluble plant protein;
d) subjecting the plant juice supernatant or plant juice permeate to ultrafiltration, optionally in diafiltration mode, to provide a soluble plant protein concentrate; and
e) subjecting the soluble plant protein concentrate to hydrophobic column adsorption. WO2014/104880A1 claims that said process is applicable on industrial scale and is economically feasible. Example 1 discloses that 40 kg of dry protein comprising 95% protein, essentially RuBisCo, was produced from about 100 m² sugar beet leaves, resulting, after disrupting and screw pressing, in 3000 kg sugar beet leaf juice. It is not clear from Example 1 how ‘100 m² sugar beet leaves’ is to be construed and how this can yield 3000 kg sugar beet leaf juice. No yield for soluble functional protein, or RuBisCo, based on kg of processed sugar beet leaves or based on sugar beet leaves obtained per hectare harvested is disclosed in Example 1.

Combined FIGS. 3 and 4A of WO2014/104880A1 disclose a flow scheme wherein 33 tonnes/hr of sugar beet leaves are processed to 15 m³/hr of sugar beet leaf juice which is subsequently converted to 330-660 kg/hr of dry protein powder. Given a typical yield of between 25 and 40 tonnes/hectare of sugar beet leaves, FIGS. 3 and 4A disclose a production of between 250 and 800 kg dry protein powder from sugar beet leaves per hectare. Given a typical RuBisCo content of between 1.5 and 2 wt. % in sugar beet leaves, the maximum theoretical production of RuBisCo (100% yield) per hectare is between 375 and 800 kg. Combined FIGS. 3 and 4A of WO2014/104880A1 therefore present a theoretical and an unrealistically high yield of dry protein powder unless the dry protein powder comprises substantial amounts of non-soluble/green proteins. FIG. 4A does, however, not disclose the constituents of the dry protein powder. WO2014/104880A1 does therefore not describe a process wherein a yield for soluble functional protein or RuBisCo based on kg of sugar beet leaves processed or based on sugar beet leaves obtained per hectare harvested is disclosed.

As shown in the appended Comparative Example 1, the current inventors have found that the process of Example 1 of WO2014/104880A1 leads to a yield of isolated soluble functional protein of less than about 27% based on the weight of the soluble functional protein present in the mash obtained after mechanical disruption of the plant material (sugar beet leaves).

Therefore, there is still a need for methods that enable isolating soluble functional plant proteins from plant material, such as sugar beet leaves, on an industrial scale in a more economical and/or efficient way. In particular, there is a need for methods for isolating soluble functional plant proteins on an industrial scale with an improved yield and with good purity.

### SUMMARY OF THE INVENTION

The present inventors have found that these objects can be met by (i) separating the aggregated chloroplast membranes from the soluble plant protein by at least two serial centrifugation steps before microfiltration such that the wet solids content is 0.5 wt. % or less before applying microfiltration, and by (ii) recycling one or more retentate streams obtained during the process.

Accordingly, in a first aspect, the invention relates to a method for isolating soluble functional plant protein from a plant material, said method comprising the following steps:
a) mechanically disrupting the cells of the plant material to obtain a mush stream Mₐ comprising plant juice and disrupted cells;
b) subjecting the mush stream Mₐ obtained in step (a) to a coarse physical separation step wherein the plant juice is separated from a pulp comprising disrupted cells, resulting in a permeate P_{b} comprising plant juice and a retentate R_{b} comprising disrupted cells, wherein the retentate R_{b} is optionally subjected to mechanical pressing resulting in a concentrated fibre stream R_{b}′ and an aqueous stream F_{b};
c) subjecting the permeate P_{b} obtained in step (b) to mild treatment at a temperature between 20° C. and 60° C. for at least 1 minute, optionally in the presence of one or more flocculants, resulting in a treated permeate P_{c} comprising aggregates or flocculates;
d) subjecting the treated permeate P_{c} obtained in step (c) to n serial centrifugation steps, wherein n is an integer ranging from 2 to 5, wherein each centrifugation step i, wherein i is an integer between 1 and n, results in a pellet fraction Xᵢ and a centrate Cᵢ, wherein the centrate Cₓ of centrifugation step x, wherein x is an integer ranging from 1 to n−1, is subjected to centrifugation in centrifugation step x+1,
   wherein the centrifugation steps are performed using disc stack centrifuges wherein the feed enters under pressure through a nozzle at the bottom of the centrifuge in the liquid phase already present in the centrifuge and is accelerated to rotor speed,
   wherein the centrate Cₙ obtained in centrifugation step n has a wet solids content of 0.5 wt. % or less, based on the total weight of the centrate Cₙ, and
   wherein any pellet fraction Xᵢ is optionally subjected to mechanical pressing resulting in a pressed pellet fraction Xᵢ′ and an aqueous stream F_{c,i};
e) subjecting centrate Cₙ obtained in centrifugation step n of step (d) to a microfiltration step resulting in a permeate Pₑ and a retentate Rₑ, wherein the retentate Rₑ is optionally subjected to mechanical pressing resulting in a pellet fraction Rₑ′ and an aqueous stream Fₑ;
f) subjecting the permeate Pₑ from microfiltration step (e) to an ultrafiltration step, optionally performed as diafiltration step, resulting in a permeate P_{f} and a retentate R_{f};
g) subjecting the retentate R_{f} obtained in step (f) to hydrophobic column adsorption to provide a column permeate P_{g} and a retentate R_{g} remaining on the static phase of the hydrophobic column; and
h) drying the column permeate P_{g} obtained in step (g) to provide a soluble functional protein isolate and water;
   wherein the method further comprises a recycling step comprising:
(AA) recycling at least part B of retentate R_{b}, or at least part Dᵢ of stream Xᵢ, wherein i is an integer selected from 1 to n, or at least part E of retentate Rₑ, or combinations thereof, to the coarse physical separation step in step (b);
(BB) when mechanical pressing is performed in any one of steps (b), (d) or (e), recycling at least part B′ of aqueous stream F_{b}, or at least part Dᵢ′ of aqueous stream F_{c,i}, wherein i is an integer selected from 1 to n, or at least part E′ of aqueous stream Fₑ, or combinations thereof, to the mild treatment step in step (c);
(CC) recycling at least part Dᵢ of stream Xᵢ, wherein i is an integer selected from 1 to n, or at least part E of retentate Rₑ, or combinations thereof, to the first centrifugation step in step (d); or
(DD) when mechanical pressing is performed in step (d) or (e), recycling at least part Dᵢ of stream Xᵢ, or at least part Dᵢ′ of aqueous stream F_{c,i}, wherein i is an integer selected from 1 to n, or at least part E of retentate Rₑ, or at least part E′ of aqueous stream Fₑ, or combinations thereof, to the first centrifugation step in step (d).

The inventors have found that this method enables the isolation of soluble functional protein on industrial scale (more than 1000 kg/h) with yields of 39-85% (based on total soluble functional protein present in the plant material or present in the mash obtained after mechanical disruption of the plant material) with purities up to 85%.

### BRIEF DESCRIPTION OF THE FIGURES

FIG. 1 schematically depicts a method for isolating soluble functional plant protein from a plant material comprising a mechanical disruption step (a), a coarse physical separation step (b), a mild treatment step (c), n serial centrifugation steps (d), a microfiltration step (e), an ultra/diafiltration step (f), a hydrophobic column adsorption step (g) and a drying step (h) to provide a soluble functional protein isolate.

FIG. 2a schematically depicts a method for isolating soluble functional plant protein from a plant material comprising a mechanical disruption step (a), a coarse physical separation step (b), a mild treatment step (c), two serial centrifugation steps (d), a microfiltration step (e), an ultra/diafiltration step (f), a hydrophobic column adsorption step (g) and a drying step (h) to provide a soluble functional protein isolate. The retentate R_{b} obtained in coarse physical separation step (b) is optionally subjected to mechanical pressing resulting in a concentrated fibre stream R_{b}′ and an aqueous stream F_{b} (dashed box and dashed arrows). Pellet fractions X₁ and X₂ obtained in centrifugation step (d) are optionally subjected to mechanical pressing resulting in a pressed pellet fractions Xᵢ′ and X₂′, and aqueous streams F_{c,1} and F_{c,2}, respectively (dashed box and dashed arrows). The retentate Rₑ obtained in microfiltration step (e) is optionally subjected to mechanical pressing resulting in a pellet fraction Rₑ′ and an aqueous stream Fₑ (dashed box and dashed arrows).

FIG. 2b schematically depicts the possible recycle streams according to the invention (with n=2 as an example). Streams (or at least part of the streams) R_{b}, X₁, X₂ and/or Rₑ can be recycled to the coarse physical separation in step (b). Streams (or at least part of the streams) F_{b}, F_{c,1}, F_{c,2} and/or Fₑ can be recycled to the mild treatment in step (c). Streams (or at least part of the streams) Rₑ, X₁, X₂, Fₑ, F_{c,1}, and/or F_{c,2} can be recycled to the first centrifugation in step (d).

FIG. 3 schematically depicts a method according to the invention for isolating soluble functional plant protein from a plant material comprising a mechanical disruption step (a), a coarse physical separation step (b), a mild treatment step (c), two serial centrifugation steps (d), a microfiltration step (e), an ultra/diafiltration step (f), a hydrophobic column adsorption step (g) and a drying step (h) to provide a soluble functional protein isolate, wherein at least parts Dᵢ and D₂ of streams X₁ and X₂, respectively, and at least part E of retentate Rₑ are recycled to the first centrifugation step in step (d).

FIG. 4 schematically depicts a method according to the invention for isolating soluble functional plant protein from a plant material comprising a mechanical disruption step (a), a coarse physical separation step (b), a mild treatment step (c), two serial centrifugation steps (d), a microfiltration step (e), an ultra/diafiltration step (f), a hydrophobic column adsorption step (g) and a drying step (h) to provide a soluble functional protein isolate, wherein pellet fractions X₁ and X₂ obtained in centrifugation step (d) are subjected to mechanical pressing resulting in pressed pellet fractions Xᵢ′ and X₂′, and aqueous streams F_{c,1} and F_{c,2}, respectively, and wherein retentate Rₑ obtained in microfiltration step (e) is subjected to mechanical pressing resulting in a pellet fraction Rₑ′ and an aqueous stream Fₑ. Part Dᵢ′ of aqueous stream F_{c,1}, part D₂′ of aqueous stream F_{c,2}, and part E′ of aqueous stream Fₑ are recycled to the first centrifugation step in step (d).

FIG. 5 schematically depicts a method according to the invention for isolating soluble functional plant protein from a plant material comprising a mechanical disruption step (a), a coarse physical separation step (b), a mild treatment step (c), two serial centrifugation steps (d), a microfiltration step (e), an ultra/diafiltration step (f), a hydrophobic column adsorption step (g) and a drying step (h) to provide a soluble functional protein isolate, wherein pellet fraction X₁ obtained in centrifugation step (d) is subjected to mechanical pressing resulting in a pressed pellet fraction Xᵢ′ and an aqueous stream F_{c,1}. Part Dᵢ′ of aqueous stream F_{c,1}, part D₂ of pellet fraction X₂ obtained in centrifugation step (d), and part E of retentate Rₑ obtained in microfiltration step (e) are recycled to the first centrifugation step in step (d).

FIG. 6 schematically depicts a method according to the invention for isolating soluble functional plant protein from a plant material comprising a mechanical disruption step (a), a coarse physical separation step (b), a mild treatment step (c), two serial centrifugation steps (d), a microfiltration step (e), an ultra/diafiltration step (f), a hydrophobic column adsorption step (g) and a drying step (h) to provide a soluble functional protein isolate, wherein retentate R_{b} is subjected to mechanical pressing resulting in a concentrated fibre stream R_{b}′ and an aqueous stream F_{b}, and wherein pellet fraction X₁ obtained in centrifugation step (d) is subjected to mechanical pressing resulting in a pressed pellet fraction Xᵢ′ and an aqueous stream F_{c,1}. Part Dᵢ′ of aqueous stream F_{c,1}, part D₂ of pellet fraction X₂ obtained in centrifugation step (d), and part E of retentate Rₑ obtained in microfiltration step (e) are recycled to the first centrifugation step in step (d). Part B′ of aqueous stream F_{b} is recycled to the mild treatment step in step (c).

FIG. 7 schematically depicts a method according to the invention for isolating soluble functional plant protein from a plant material comprising a mechanical disruption step (a), a coarse physical separation step (b), a mild treatment step (c), two serial centrifugation steps (d), a microfiltration step (e), an ultra/diafiltration step (f), a hydrophobic column adsorption step (g) and a drying step (h) to provide a soluble functional protein isolate, wherein retentate R_{b} is subjected to mechanical pressing resulting in a concentrated fibre stream R_{b}′ and an aqueous stream F_{b}, and wherein pellet fraction X₁ obtained in centrifugation step (d) is subjected to mechanical pressing resulting in a pressed pellet fraction Xᵢ′ and an aqueous stream F_{c,1}. Part D₂ of pellet fraction X₂ obtained in centrifugation step (d) and part E of retentate Rₑ obtained in microfiltration step (e) are recycled to the first centrifugation step in step (d). Part B′ of aqueous stream F_{b} and part Dᵢ′ of aqueous stream F_{c,1} are recycled to the mild treatment step in step (c).

FIG. 8 schematically depicts a method according to the invention for isolating soluble functional plant protein from a plant material comprising a mechanical disruption step (a), a coarse physical separation step (b), a mild treatment step (c), two serial centrifugation steps (d), a microfiltration step (e), an ultra/diafiltration step (f), a hydrophobic column adsorption step (g) and a drying step (h) to provide a soluble functional protein isolate, wherein pellet fractions X₁ and X₂ obtained in centrifugation step (d) are subjected to mechanical pressing resulting in a pressed pellet fractions Xᵢ′ and X₂′, and aqueous streams F_{c,1} and F_{c,2}, respectively, wherein retentate Rₑ obtained in microfiltration step (e) is subjected to mechanical pressing resulting in a pellet fraction Rₑ′ and an aqueous stream Fₑ, and wherein retentate R_{b} is subjected to mechanical pressing resulting in a concentrated fibre stream R_{b}′ and an aqueous stream F_{b}. Part B′ of aqueous stream F_{b}, part Dᵢ′ of aqueous stream F_{c,1}, part D₂′ of aqueous stream F_{c,2}, and part E′ of aqueous stream Fₑ are recycled to the mild treatment step in step (c).

FIG. 9 schematically depicts a method according to the invention for isolating soluble functional plant protein from a plant material comprising a mechanical disruption step (a), a coarse physical separation step (b), a mild treatment step (c), two serial centrifugation steps (d), a microfiltration step e), an ultra/diafiltration step (f), a hydrophobic column adsorption step (g) and a drying step (h) to provide a soluble functional protein isolate, wherein at least parts D₁ and D₂ of streams X₁ and X₂, respectively, at least part E of retentate Rₑ, and at least part B of retentate R_{b} are recycled to the coarse physical separation step in step (b).

FIGS. 10a-10d schematically depict methods according to the invention for isolating soluble functional plant protein, as applied in Examples 2-5.

FIG. 11 shows the turbidity of the treated permeate P_{c}, i.e. after mild treatment step (c), and of the centrates C₁ and C₂ after a first centrifuging step (d) with a decanter centrifuge and a second centrifuging step (d) with a disc stack centrifuge positioned in series.

FIG. 12 shows a (volume-based) particle size distribution in the treated permeate P_{c} and in the centrates C₁ obtained after centrifugation with different disc stack centrifuges.

### DETAILED DESCRIPTION

Accordingly, in a first aspect of the invention a method is provided for isolating soluble functional plant protein from a plant material, said method comprising the following steps:
a) mechanically disrupting the cells of the plant material to obtain a mush stream Mₐ comprising plant juice and disrupted cells;
b) subjecting the mush stream Mₐ obtained in step (a) to a coarse physical separation step wherein the plant juice is separated from a pulp comprising disrupted cells, resulting in a permeate P_{b} comprising plant juice and a retentate R_{b} comprising disrupted cells, wherein the retentate R_{b} is optionally subjected to mechanical pressing resulting in a concentrated fibre stream R_{b}′ and an aqueous stream F_{b};
c) subjecting the permeate P_{b} obtained in step (b) to mild treatment at a temperature between 20° C. and 60° C. for at least 1 minute, optionally in the presence of one or more flocculants, resulting in a treated permeate P_{c} comprising aggregates or flocculates;
d) subjecting the treated permeate P_{c} obtained in step (c) to n serial centrifugation steps, wherein n is an integer ranging from 2 to 5, wherein each centrifugation step i, wherein i is an integer between 1 and n, results in a pellet fraction Xᵢ and a centrate Cᵢ, wherein the centrate Cₓ of centrifugation step x, wherein x is an integer ranging from 1 to n−1, is subjected to centrifugation in centrifugation step x+1,
   wherein the centrate Cₙ obtained in centrifugation step n has a wet solids content of 0.5 wt. % or less, based on the total weight of the centrate Cₙ, and
   wherein any pellet fraction Xᵢ is optionally subjected to mechanical pressing resulting in a pressed pellet fraction Xᵢ′ and an aqueous stream F_{c,i};
e) subjecting centrate Cₙ obtained in centrifugation step n of step (d) to a microfiltration step resulting in a permeate Pₑ and a retentate Rₑ, wherein the retentate Rₑ is optionally subjected to mechanical pressing resulting in a pellet fraction Rₑ′ and an aqueous stream Fₑ;
f) subjecting the permeate Pₑ from microfiltration step (e) to an ultrafiltration step, optionally performed as diafiltration step, resulting in a permeate P_{f} and a retentate R_{f};
g) subjecting the retentate R_{f} obtained in step (f) to hydrophobic column adsorption to provide a column permeate P_{g} and a retentate R_{g} remaining on the static phase of the hydrophobic column; and
h) drying the column permeate P_{g} obtained in step (g) to provide a soluble functional protein isolate and water;
   wherein the method further comprises a recycling step selected from:
(AA) recycling at least part B of retentate R_{b}, at least part Dᵢ of stream Xᵢ, wherein i is an integer selected from 1 to n, at least part E of retentate Rₑ, or combinations thereof, to the coarse physical separation step in step (b);
(BB) when mechanical pressing is performed in any one of steps (b), (d) or (e), recycling at least part B′ of aqueous stream F_{b}, at least part Dᵢ′ of aqueous stream F_{c,i}, wherein i is an integer selected from 1 to n, at least part E′ of aqueous stream Fₑ, or combinations thereof, to the mild treatment step in step (c);
(CC) recycling at least part Dᵢ of stream Xᵢ, wherein i is an integer selected from 1 to n, at least part E of retentate Rₑ, or combinations thereof, to the first centrifugation step in step (d); or
(DD) when mechanical pressing is performed in any one of steps (b), (d) or (e), recycling at least part Dᵢ of stream Xᵢ, at least part Dᵢ′ of aqueous stream F_{c,i}, wherein i is an integer selected from 1 to n, at least part E of retentate Rₑ, at least part E′ of aqueous stream Fₑ, or combinations thereof, to the first centrifugation step in step (d).

The word ‘soluble’ in ‘soluble functional protein’ and ‘soluble functional protein isolate’ as used herein refers to ‘aqueous solubility’.

The term ‘wet solids content’ in the context of centrate Cₙ obtained in step (d) concerns the phase of undissolved solids that can be separated from the liquid in the centrate Cₙ using a ‘universal’ high-speed centrifuge. This solid phase is still wet because the solids still contain some water that cannot be removed using centrifuging. The wet solids content [wt. %], based on the total weight of the centrate Cₙ, as used and defined herein, is measured as follows:
1) Provide a 15 mL test tube and measure its weight (Mass1);
2) Pipette about 10 g of a sample of the centrate Cₙ into the test tube and measure the total weight of the sample and the test tube (Mass2);
3) Centrifuge the sample of the centrate Cₙ for 60 min. at 1500 g (Hermle Z323 centrifuge, 3000 rpm);
4) Separate the liquid from the pellet fraction (the wet solids), e.g. by putting the test tube upside down and letting the liquid drain from the pellet;
5) Measure the total weight of the test tube with pellet (Mass3); and
6) Determine the wet solids content of the centrate Cₙ [wt. %], based on the total weight of the centrate Cₙ, with the following formula:

$$( \frac{M a s s 3 - M a s s 1}{M a s s 2 - M a s s 1} ) * 100 \%$$

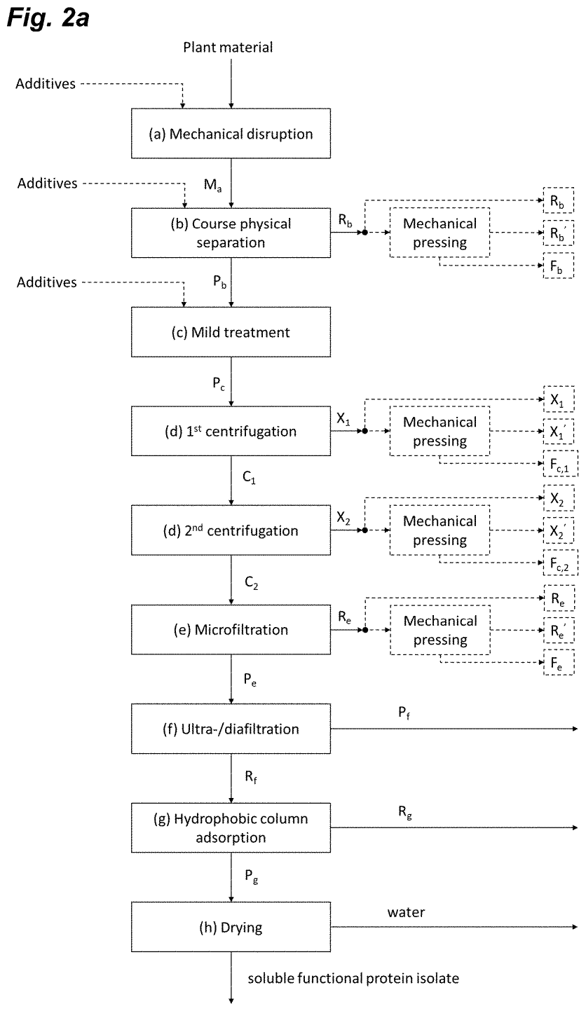

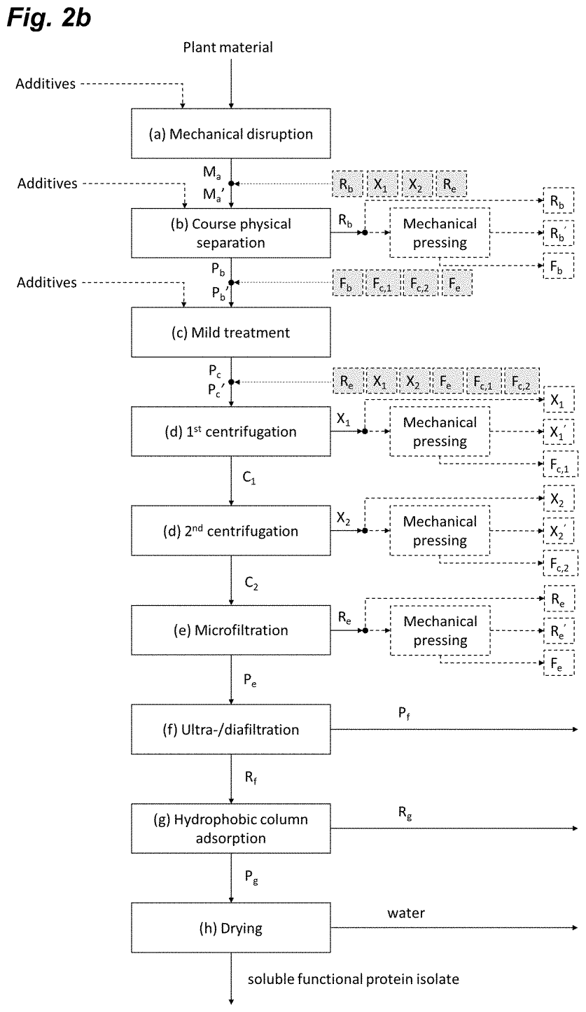

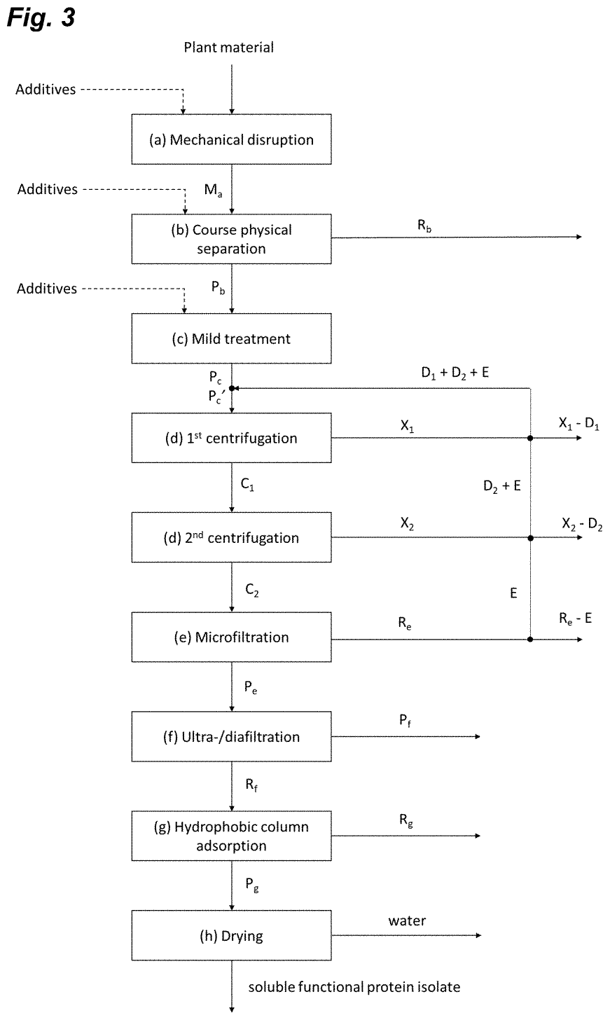

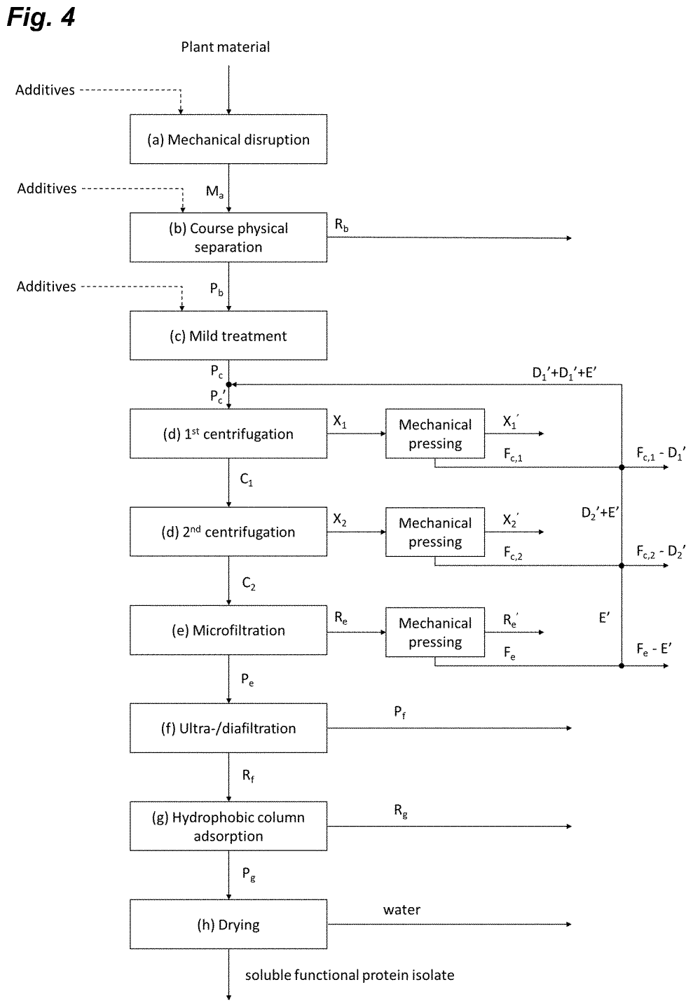

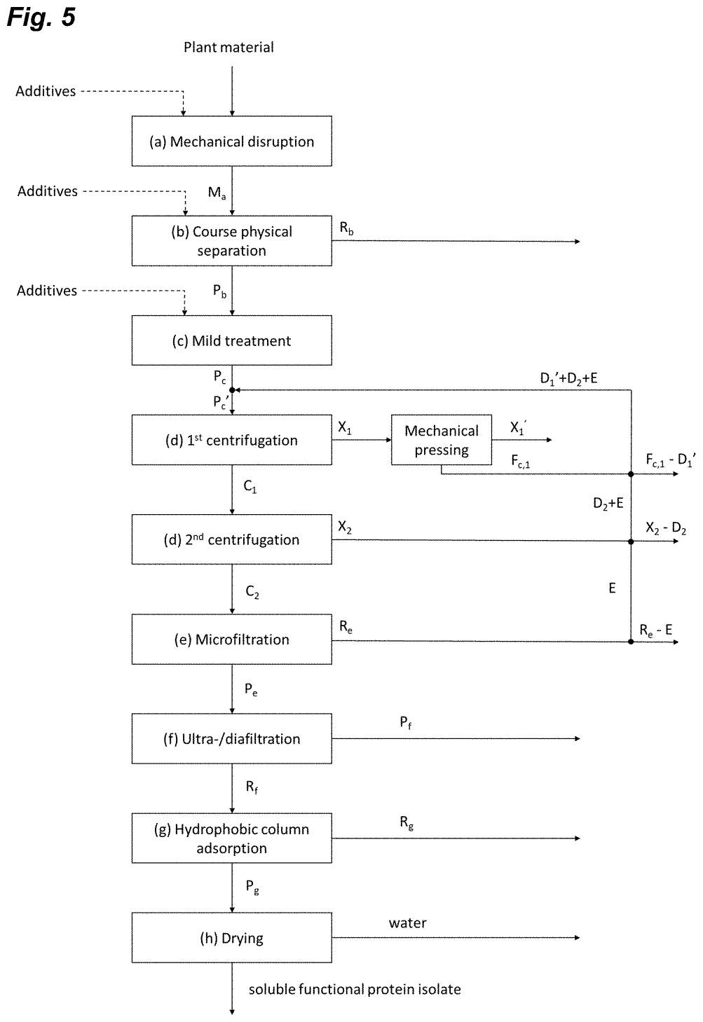

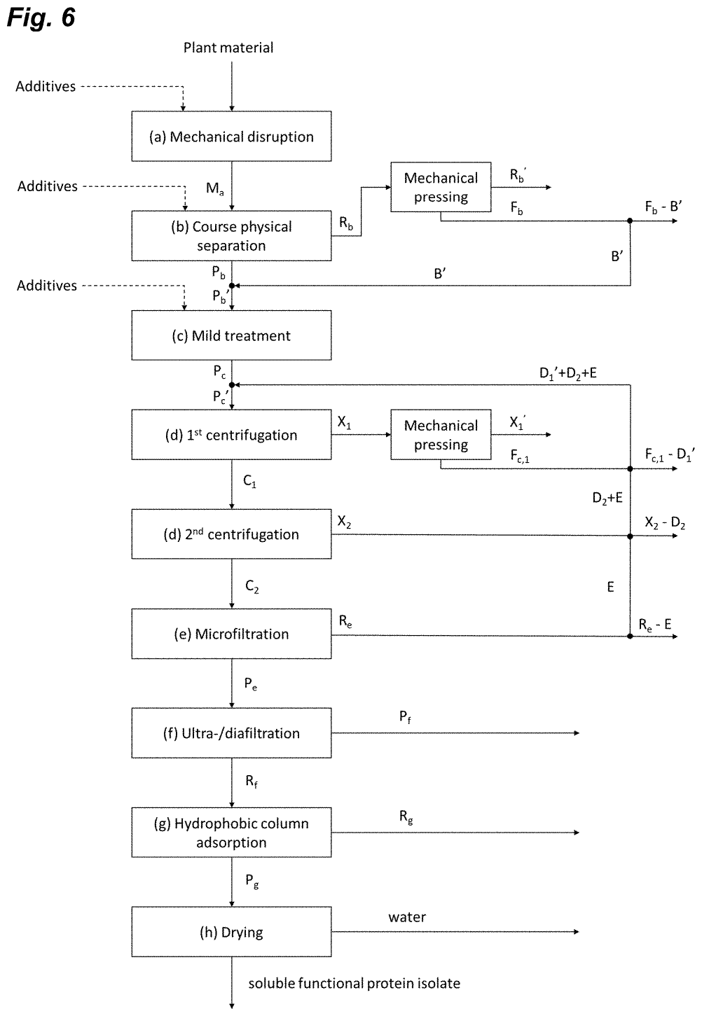

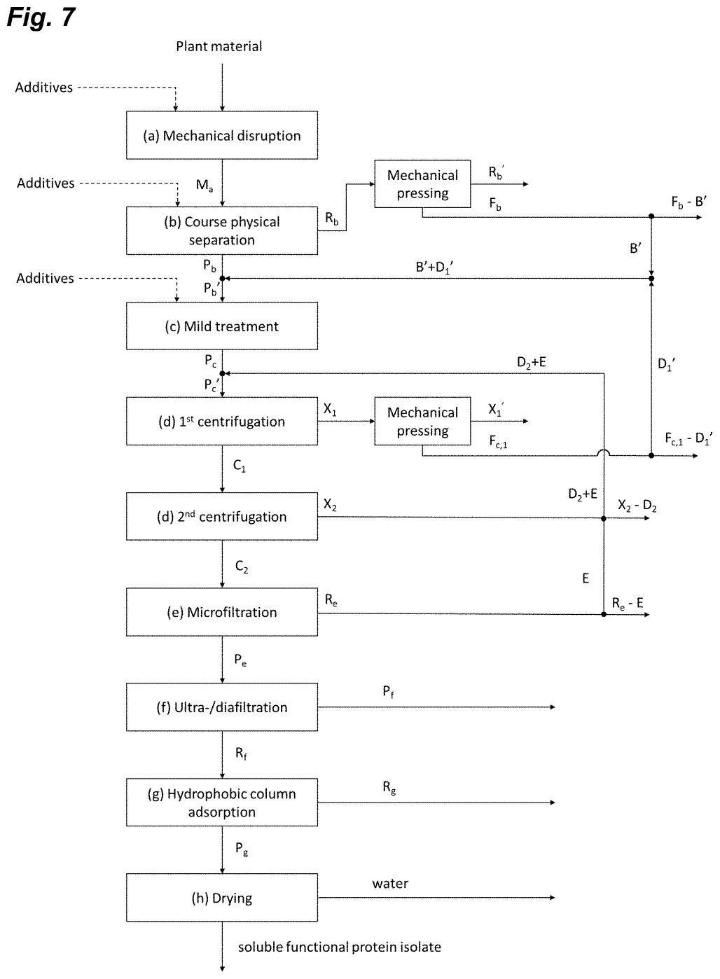

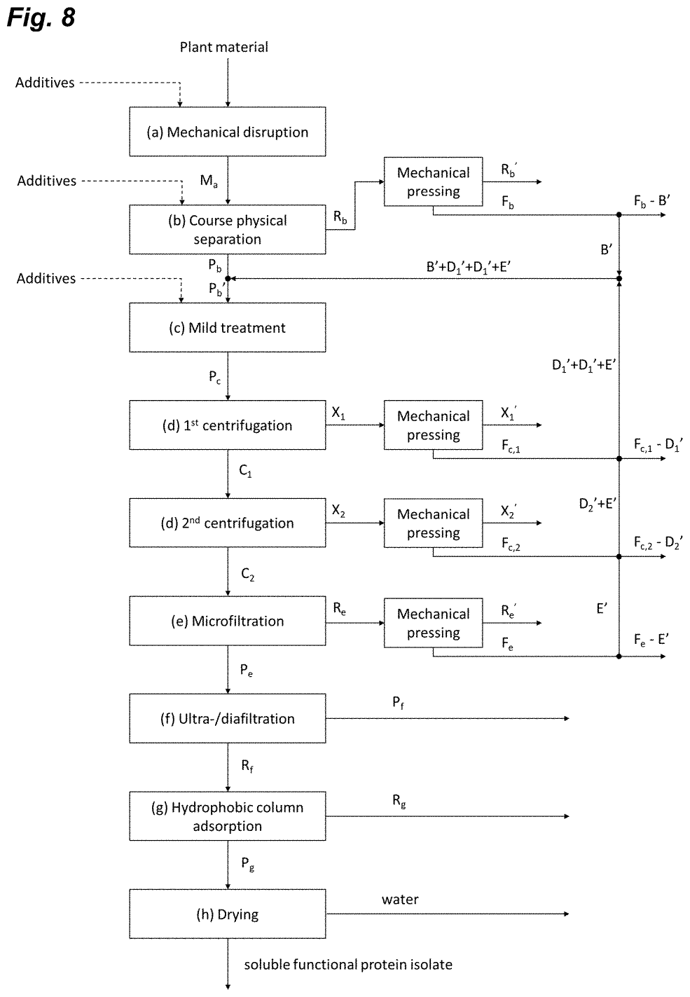

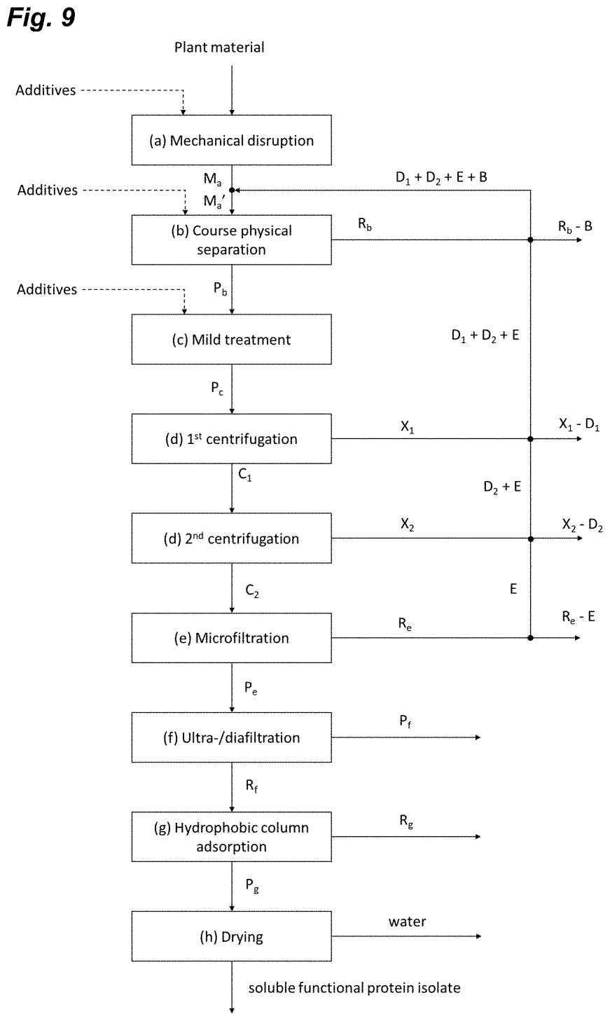

If a stream is recycled to the coarse physical separation step in step (b), the volume of the feed entering the coarse physical separation step in step (b) is changed. Initially, mush stream Mₐ is fed to the coarse physical separation step in step (b). If this mush stream is combined with a recycle stream, the resulting mush stream is indicated herein with Mₐ′. See for example FIG. 2b.

If a stream is recycled to the mild treatment step in step (c), the volume of the feed entering the mild treatment step in step (c) is changed. Initially, permeate P_{b} is fed to the mild treatment step in step (c). If this permeate is combined with a recycle stream, the resulting permeate is indicated herein with P_{b}′. See for example FIG. 2b.

If a stream is recycled to the first centrifugation step in step (d), the volume of the feed entering the first centrifugation step in step (d) is changed. Initially, permeate P_{c} is fed to the first centrifugation step in step (d). If this permeate is combined with a recycle stream, the resulting permeate is indicated herein with P_{c}′. See for example FIG. 2b.

In a preferred embodiment of the invention the plant material comprises or consists of green leaves, wherein the green leaves preferably comprise more than 5 wt. % of dry matter, more preferably more than 10 wt. %, even more preferably more than 15 wt. %, still more preferably more than 18 wt. %, and most preferably more than 20 wt. %. In another preferred embodiment, the plant material comprises or consists of green leaves, wherein the green leaves comprise between 5 wt. % and 25 wt. % of dry matter, more preferably between 10 wt. % and 20 wt. %, even more preferably between 12 wt. % and 18 wt. %.

Dry matter content and protein content in juice obtained by twin screw pressing of green leaves was analyzed for different types of lettuce, different of types of endive, sugar beet leaves and alfalfa (see Table 1). The present inventors have found that there is a strong correlation between total soluble protein content and dry matter content in green leaves for green leaves of different origin. This means that the technology described herein for the separation of soluble functional proteins from dry matter is in general applicable to different types of green leaves.

TABLE 1
Dry matter and soluble protein in different green leaves

| | Estimated dry | Soluble protein |
|---|---|---|
| | matter content | content in the |
| | (wt. %) | juice* (wt. %) |
| Lettuce | | |
| Batavia lettuce | 4 | 0.2 |
| Butterhead | 4 | 0.2 |
| Iceberg | 3 | 0.02 |
| Endive | | |
| Escarole | 7 | 0.3 |
| Curly Endive | 5 | 0.1 |
| Sugar beet leaves | 16 | 1.2 |
| Alfalfa | 21 | 2.4 |
| Chicory leaves | 13 | 0.5 |
| Leaf radish | 13 | 0.3 |
| Rye grass | 16 | 1.8 |
| Carrot leaves | 6 | 0.2 |

*the soluble protein content in the juice was measured before heating with Bradford analysis

In a very preferred embodiment, the plant material comprises, preferably consists of, the green leaves of sugar beet, alfalfa, chicory, fodder chicory, phacelia, ryegrass, rye, oat, radish, fodder radish, vetches, carrot leaf, chicory leaf, and combinations thereof. Most preferably, the plant material comprises, preferably consists, of sugar beet green leaves.

### Step (a) Mechanical Disruption

In a first step, the cells of the plant material are mechanically disrupted to form a mush comprising plant juice and disrupted cells. This process is used to release the intracellular plant juice from the cells of the plant material.

Mechanical disruption to form a mush is preferably performed by:
screw press homogenization, i.e. using a screw press wherein solids and liquids are not separated;
milling, such as hammer milling;
pulsed electric field treatment;
crushing;
slicing; or
combinations thereof.

In an embodiment, at least one reducing agent may be added before, during or after step (a). The reducing agent is intended to limit or prevent oxidation of the protein to be isolated during the remainder of the isolation and purification process. Examples of reducing agents that can be applied in the method according to the invention are lithium aluminum hydride (LiAlH₄), nascent (atomic) hydrogen, sodium amalgam, sodium borohydride (NaBH₄), compounds containing the Sn²⁺ ion, such as tin(II) chloride, sulfite compounds, hydrazine, diisobutylaluminum hydride (DIBAL), lindlar catalyst, oxalic acid (C₂H₂O₄), formic acid (HCOOH), ascorbic acid (C₆H₈O₆), phosphites, hypophosphites, phosphorous acid (H₃PO₄), dithiothreitol (DTT), and compounds containing the Fe²⁺ ion, such as iron(II) sulfate, metal hydrides such as NaH, CaH₂, and LiAlH₄, active metals such as sodium, magnesium, aluminium and zinc, ADH, alcohol dehydrogenase, boranes, catecholborane, copper hydride, copper, diborane, diethyl 1,4-dihydro-2,6-dimethyl-3,5-pyridinecarboxylate, diisopropylaminoborane, dimethylsulfide borane, Fe, formaldehyde, Hantzsch ester, hydrogen, iron, isopropanol, lithium, lithium aluminum hydride, lithium, magnesium, manganese, mercaptopropionic acid, 2-nitrobenzenesulfonylhydrazide, phenylsilane, pinacolborane, polymethylhydrosiloxane, potassium, potassium iodide, 2-propanol, Red-A1, silanes, sodium, sodium borohydride, sodium cyanoborohydride, sodium dithionite, sodium hydrosulfite, sodium hydroxymethanesulfinate, sodium tetrahydroborate, sodium triacetoxyborohydride, tetramethyldisiloxane, trichlorosilane, triethylphosphine, trimethylphoshpine, triphenylphosphine, triphenylphosphite, triethylsilane, tris(trimethylsilyl)silane, bisulfite salts, cysteine, or combinations thereof. Preferably, the at least one reducing agent is food grade.

In an embodiment, the at least one reducing agent is selected from bisulfite salts, such as alkali cation bisulfite salts, more particularly sodium bisulfite. Bisulfite salts act as a preservative and antioxidant and reduce browning reactions.

Foam may be generated during the mechanical disruption step (a). Foam formation may result in unfolding of protein so that polyphenols can bind covalently to the soluble functional protein and result in loss of the soluble functional protein during the isolation process. Hence, foam formation is not desired in the process of the present invention. Hence, an anti-foaming agent may be added before, during or after step (a).

In an embodiment of the invention, the plant juice obtained in the mechanical disruption step (a) has a pH of about 6-8, preferably 6-7 and more preferably around 6.5. The solubility of certain proteins, such as RuBisCo, is high at this pH. Higher pH values result in auto-oxidation of polyphenols and binding of certain proteins, such as RuBisCo, to thylakoid membranes. The pH may be adjusted using acid or base. Acid or base may be added before, during or after step (a) to establish said pH.

In an embodiment, acid or base is added before, during or after step (a), preferably after adding the at least one reducing agent, to establish said pH of the plant juice obtained in the mechanical disruption step of about 6-8, preferably 6-7 and more preferably around 6.5.

In an embodiment, the pH is adjusted to be within the above range after adding bisulfite salts using NaOH.

As will be appreciated by those skilled in the art, oxidation reactions can be limited or prevented if the material to be processed is not contacted with oxidative compounds such as molecular oxygen during the processing.

The inventors have established that oxidation reactions and browning can be effectively reduced or prevented by working under an inert atmosphere. Accordingly, the process as defined herein is preferably performed under an inert atmosphere, preferably under an atmosphere of molecular nitrogen (N₂).

In a very preferred embodiment, before, during or after step (a), preferably before step (a), the molecular oxygen (O₂) in the material to be processed is displaced with molecular nitrogen (N).

### Step (b) Coarse Physical Separation Step

In a next step the mush stream Mₐ obtained in step (a) is subjected to a coarse physical separation step wherein plant juice is separated from a pulp comprising disrupted cells. In the art, this process is also referred to as extraction of the liquid plant juice from the disrupted cells. As will be appreciated by those skilled in the art, a high plant juice yield with a low wet solids content is the preferred result.

In a very preferred embodiment, the coarse physical separation step is performed as a continuous process.

This process is preferably performed using a dewatering press or filter press, such as a dewatering screw press or screw filter press. A dewatering press or filter press as defined herein is a press that can separate liquids from solids by squeezing the mush against a filter element such as a screen, filter or sieve, and collecting the plant juice through the filter element. The dewatering press or filter press as used herein preferably operates on the principle of cross-flow filtration.

In a preferred embodiment, the coarse physical separation step is performed using a dewatering press with a rotating hydraulic piston-cylinder system.

The mush is preferably forced tangentially across the surface of the filter element and the permeable components (the juice and particles smaller than the pores, holes or sieve openings) cross the filter element.

In preferred embodiments, the dewatering press or filter press is a dewatering screw press or screw filter press and the mush is forced tangentially across the surface of the filter element by a rotating screw element such that the permeable components (the juice and particles smaller than the pores, holes or sieve openings) cross the filter element. The larger particles remain on the filter element to form a cake with a certain residual moisture content. The rotating screw element pushes the mush and the cake formed on the filter element over the entire surface of the filter element. The mush is thus split into two fractions: a permeate P_{b} comprising the plant juice and a pulp stream or retentate R_{b} comprising the disrupted cells. Preferably, the screw element is a screw pile comprising a shaft and one or more helical threads. The number of helical threads on the shaft defines the number of channels through which the screw element pushes the mush and the cake formed on the filter element over the entire surface of the filter element. The number of helical threads on the shaft typically ranges between 1 and 8, such as 1, 2, 4, 6 or 8. In preferred embodiments, the number of helical threads on the shaft is 2, defining two channels, spaced 1800 apart, through which the screw element pushes the mush and the cake formed on the filter element over the entire surface of the filter element. In another preferred embodiments, the number of helical threads on the shaft is 4, defining four channels, spaced 900 apart, through which the screw element pushes the mush and the cake formed on the filter element over the entire surface of the filter element. In preferred embodiments, the pitch size (the distance between helical threads in upstream direction, is between 5 and 20 cm. In preferred embodiments, the dewatering screw press or screw filter press comprises a screw pile comprising a shaft and one or more helical threads of constant diameter placed in a conical filter unit such that the distance between the screw pile and the filter unit is constant and has a value of between 0 and 20 mm, such as 0 mm, 2 mm, 6 mm or 12 mm. In preferred embodiments, the most upstream part of the filter unit, just before a discharge pipe for the pressed cake, is air tight, i.e. not perforated. It will be understood by the skilled person that the length and diameter of the screw pile and the diameter of the helical threads can be varied to obtain the maximal amount of plant juice. In embodiments, the rotation speed of the screw pile is between 15 and 60 Hz, preferably between 20 and 60 Hz, more preferably between 25 and 60 Hz. In embodiments, the height of the helical threads (the difference between the diameter of the helical threads and the shaft diameter) is between 1.5 and 20 mm, more preferably between 1.6 and 15 mm, such as between 2 mm, 4 mm, 6 mm, 8 mm, 10 mm or 12 mm.

In preferred embodiments, the filter element is a screen, filter or sieve having openings between 90 and 800 μm, more preferably between 90 and 650 μm, even more preferably between 90 and 550 μm, such as 100, 300 or 500 μm.

The yield of the permeate can be increased by applying a negative pressure or vacuum to the permeate-side of the filter element. Applying a negative pressure or vacuum to the permeate-side of the filter element can also be advantageously used to degas the permeate P_{b}. The inventors have found that molecular oxygen, present in the plant juice, accelerates oxidation reactions in downstream processing. The accelerated oxidation negatively influences the protein quality, as it has an adverse effect on the colour and taste.

Accordingly, in a preferred embodiment, the pressure or vacuum applied on the permeate-side of the filter element is between 100 and 250 mbar, resulting in a pressure difference of about 750 to about 900 mbar across the filter element. In a more preferred embodiment, the pressure or vacuum applied on the permeate-side of the filter element is between 120 and 220 mbar, such as about 200 mbar, resulting in a pressure difference of about 780 to about 880 mbar across the filter element, such as about 800 mbar.

In an embodiment, a dewatering screw press or screw filter press as defined herein is used, wherein the screw press separates the liquids from the solids by squeezing the mush using a screw pile comprising a shaft and one or more helical threads against a filter element, such as a screen filter or sieve, by collecting the plant juice through the filter element and by applying a negative pressure or vacuum to the permeate-side of the filter element, wherein the rotation speed of the screw pile is between 25 and 60 Hz, the filter element is a screen, filter or sieve having openings between 90 and 600 μm, the number of helical threads is 2 and the height of the helical threads is between 1.5 and 15 mm.

As will be appreciated by those skilled in the art, the different parameters for separating the plant juice from the pulp comprising disrupted cells using the dewatering press or filter press (filter size, screw size, number of helical threads and screw speed) can be varied depending on the type of plant material to obtain a maximal plant juice yield.

In highly preferred embodiments, the dewatering screw press or screw filter press as used herein is an hydraulic press with a piston-cylinder system (such as HP1600-TS, HPX 6007, HPX 7507 or HPX 12007) from Bucher Unipektin AG, an UDE screw press from Bruckner Liquid Food Tech GmbH, an Extruder B55 from Lehman or a cylindrical screw press from Ponndorf GmbH or a twin-screw press from Babbini. Preferred embodiments of the dewatering screw press or screw filter press as used herein are described in EP3321079A1, which is incorporated herein by reference in its entirety.

The inventors have established that the cake formed on the filter element which is expelled from the screw press as a pulp stream or retentate R_{b} still contains considerable amounts of soluble functional proteins, for example soluble protein in the residual moisture content of the pulp stream or soluble protein that was not released during mechanical disruption step (a).

Accordingly, in preferred embodiments, the retentate R_{b} obtained in step (b) is subjected to mechanical pressing resulting in a concentrated fibre stream R_{b}′ and an aqueous stream F_{b}. This aqueous stream F_{b} comprising soluble functional proteins can then be recycled (partly or completely) to the purification process, for example to the mild treatment step in step (c).

The permeate P_{b} typically comprises, in addition to the soluble functional proteins, chlorophyll, chlorophyll-related proteins, membrane fragments, polyphenols and other unwanted compounds. The permeate P_{b} is typically referred to in the art as a ‘green juice’.

In a preferred embodiment, permeate P_{b} is subjected to a decanting step before it is subjected to mild treatment step (c) in order to reduce its solids content. The decanted solid fraction can then be recycled to the coarse physical separation step in step (b), optionally combined with mechanical pressing of the resulting retentate. In another preferred embodiment, permeate P_{b} is subjected to a step of removal of sand and pebbles, for example using a sedimentation step or a hydrocycloning step, before it is subjected to the mild treatment step in step (c). The decanting step and the step of removal of sand and pebbles before subjecting permeate P_{b} to the mild treatment step in step (c) can advantageously be combined in a single step.

### Step (c) Mild Treatment

As is generally known in the art, proteins in green leaves precipitate at different temperatures, which can be utilized to separate the green chlorophyll-related proteins from the soluble functional proteins. Chlorophyll-related proteins in the green juice, and along with it associated membrane fractions and some of the chlorophyll, aggregate at temperatures ranging between 50 and 65° C., and the soluble functional proteins at temperatures between 64 and 80° C. at high ionic strength. Thermal treatment of extracted juice at 60° C. for 20 seconds by steam injection was for example shown to be enough to cause coagulation of the green fraction, while milder treatments at lower temperatures require more time, e.g. 50° C. for 30 minutes. In this respect, reference is made to A.-L Nynäs, (2018), White proteins from green leaves in food applications, A literature study, Alnarp: Sveriges lantbruksuniversitet (Introductory paper at the Faculty of Landscape Architecture, Horticulture and Crop Production Science, 2018:1), to A. H. Martin et al., J. Agric. Food Chem., 62 (2014), pp 10783-10791, and to R. H. Edwards, J. Agric. Food Chem., 23 (1975), pp 620-626.

Heat treatment at too high a temperature and/or for too long a period causes the protein to denaturate and destroys the functionality thereof. Accordingly, any thermal step should be as mild as possible in order to retain the functionality of the soluble protein.

In step (c), the permeate P_{b} obtained in step (b) is subjected to mild treatment, optionally in the presence of one or more flocculants, to cause aggregation or flocculation of green chlorophyll-related proteins at a temperature between 20° C. and 60° C. for at least 1 minute. As explained hereinbefore, the process according to the invention is a process aimed at keeping the extracted soluble functional protein in solution until the soluble functional protein is dried. Within this temperature range, the white or soluble functional proteins remain in solution.

Preferably said mild treatment comprises exposing the extracted juice to a temperature of between 20 and 55° C., more preferably between 25 and 55° C., even more preferably between 30 and 50° C., still more preferably between 40 and 50° C.

Divalent or trivalent cations greatly enhance the heat-induced precipitation of thylakoid membranes through flocculation, thereby effectively removing the chlorophyll from the green juice. Thus, adding flocculants such as divalent or trivalent cations may improve the yield of the isolation process of soluble function protein and the overall process speed.

Accordingly, in preferred embodiments, one or more flocculants chosen from the group consisting of salts comprising divalent cations or trivalent cations are added to the permeate P_{b} obtained in step (b) before or during step (c). By a divalent cation is meant a cation with 2 valences. By a trivalent cation is meant a cation with 3 valences. Suitable divalent cations include but are not limited to calcium (II) (Ca²⁺), magnesium (II) (Mg²⁺), beryllium (II) (Be²⁺), zinc (II) (Zn²⁺), copper (II) (Cu²⁺), iron (II) (Fe²⁺), nickel (II) (Ni²⁺), tin (II) (Sn²⁺), and barium (II) (Ba²⁺). Trivalent cations include but are not limited to boron (III) (B³⁺), aluminum (III) (Al³⁺), and iron (III) (Fe³⁺). As will understood by the skilled person, the divalent or trivalent ions are added as a salt to the permeate P_{b} obtained in step (b) before or during step (c).

The salt comprises the divalent or trivalent cation and one or more counter anions. The choice of the counter anion is not particularly limited as long as the salt is sufficiently soluble in water. In preferred embodiments, the one or more counter anions are chosen from chloride (Cl⁻) and sulfate (SO₄²⁻). In embodiments, the counter anion is an organic anion. Examples of organic anions include but are not limited to acetate and lactate. Preferably the salts used as flocculants are water soluble and food grade.

Preferred flocculants are chosen from the group consisting of calcium (II) (Ca²⁺) salts or aluminum (III) (Al³⁺) salts and combinations thereof. Examples of flocculants include, but are not limited to Ca²⁺-salts or Al³⁺-salts selected from the group consisting of CaCl₂, Ca(NO₃)₂, AlCl₃, Al(NO₃)₃, and combinations thereof.

The presence of divalent or trivalent cations will result in such efficient flocculation of the chlorophyll containing membranes that mild treatment step (c) can occur at lower temperatures.

Accordingly, in a preferred embodiment, one or more flocculants chosen from the group consisting of salts comprising divalent cations or trivalent cations as defined hereinbefore are added to the permeate P_{b} obtained in step (b) before or during step (c) and the temperature during step (c) is between 20° C. and 55° C., more preferably between 20 and 50° C., even more preferably between 20 and 40° C., still more preferably between 20 and 30° C.

Preferably, the mild treatment of step (c) is only short, such as between 1 minute and 3 hours, preferably between 1 minute and 1 hour, more preferably between 5 and 50 minutes, even more preferably between 10 and 30 minutes, still more preferably between 15 and 25 minutes, such as about 20 minutes.

If performed at temperatures of between 25 and 55° C., the mild treatment of step (c) is preferably followed by a rapid cooling of the heated juice, such as for instance cooling within 1 minute to 1 hours, more preferably within 1-30 minutes, more preferably within 1-10 minutes, to ambient temperature, or if needed, even to lower temperatures.

Hence, after the mild treatment of step (c), the juice is preferably cooled, preferably by forced cooling. Preferably the juice is cooled in less than 60 minutes, preferably less than 30 minutes, more preferably less than 10 min, 9 min, 8 min, 7 min, 6 min, 5 min, 4 min, 3 min, 2 min, or 1 minute to a temperature of about 20° C., more preferably to a temperature of about 15° C., 10° C., 9° C., 8° C., 7° C., 6° C., 5° C., 4° C., 3° C., 2° C., or 1° C. in that time frame.

Step (c) results in a treated green juice, which is called treated permeate P_{c} in what follows, comprising aggregates or flocculates typically having a size of 1 mm and larger. Aggregates or flocculates having this size can effectively be separated off using centrifugation.

### Step (d) Centrifugation

The treated permeate P_{c} obtained in step (c) thus comprises, in addition to the soluble functional proteins, an at least partly aggregated or flocculated green fraction comprising chlorophyll, chlorophyll-related proteins, membrane fragments, polyphenols and aggregates thereof. The aggregates or flocculates typically have a size of 1 mm and larger.

The treated permeate P_{c} obtained in step (c) is subjected in step (d) to centrifugation to remove at least part of the aggregates or flocculates as a pellet fraction. In addition to the pellet fraction, centrifugation results in a centrate.

Centrifugation is a mechanical process that utilizes an applied centrifugal force field to separate the components based on a difference in density and/or particle size.

Centrifugation as used herein can be performed as a batch, semi-continuous or continuous process. In embodiments, the centrifugation can be performed with decanter centrifuges, sedicanters, disc stack centrifuges or combinations thereof.

In a preferred embodiment, the centrifugation is performed as a continuous process. Examples of centrifuges that can be applied in a continuous process are continuous decanter centrifuges, continuous sedicanters, continuous disc stack centrifuges and combinations thereof. Such centrifuges continuously discharge wet pellet fractions or discharge them at regular intervals.

The inventors have established that the yield of the separation in centrifugation step (d), i.e. the degree of separation of aggregates or flocculates prepared in mild treatment step (c) from the treated permeate P_{c}, is adversely affected by high turbulence due to for example gas bubbles being present in the treated permeate P_{c}. This high turbulence results in high shear forces at the gas-liquid interface causing the aggregates or flocculates prepared in mild treatment step (c) to be disrupted and to fall apart into smaller aggregates or flocculates having for example sizes between 0.01 and 100 m. As explained hereinbefore, the aggregates or flocculates obtained in step (c) typically have a size of 1 mm and larger. Aggregates or flocculates having this size can effectively be separated off using centrifugation. It is however difficult to separate off aggregates or flocculates having a size between 0.01 and 100 m in a centrifuge and particles of such size will therefore remain in the centrate. The centrate is further purified with a microfiltration and an ultrafiltration step. The membranes used in the microfiltration and ultrafiltration steps can be easily clogged by small aggregates or flocculates having a size between 0.01 and 100 m present in the centrate of the centrifugation step, resulting in an insufficient fractionation and high protein retention. Alternatively, the membranes used in the microfiltration and ultrafiltration steps need to be cleaned too often resulting in a less efficient process. High turbulence in the centrifugation step is therefore to be avoided.

The inventors have further established that the use of a decanter centrifuge results in an inefficient separation of aggregates or flocculates prepared in the mild treatment step (c). A decanter centrifuge creates high turbulence when for example gas bubbles are present in the treated permeate P_{c}. This high turbulence results in high shear forces at the gas-liquid interface causing the aggregates or flocculates prepared in mild treatment step (c) to be disrupted and to fall apart into smaller aggregates or flocculates. Moreover, the inventors have established that, if coarse physical separation step (b) is performed according to the embodiments described hereinbefore, the permeate P_{b} no longer contains substantial amount of large particles such that a decanting step can be dispensed with. Hence, step (d) is preferably not performed using (continuous) decanter centrifuges, (continuous) sedicanters or decanters. Hence, in a very preferred embodiment, step (d) does not involve the use of decanter centrifuges, sedicanters or decanters. In an even more preferred embodiment of the process as defined herein, no decanter centrifuges, sedicanters or decanters are used after mild treatment step (c).

Preferably, the centrifuges are low-shear centrifuges. In another preferred embodiment, centrifugation step (d) is performed at low-shear conditions. Centrifugation is preferably performed between 10000 and 20000 rpm, such as 16000 rpm, for a period between 30 seconds and 2 minutes, more preferably between 40 seconds and 1.5 minutes, such as about 1 minute.

In a preferred embodiment, the centrifuges are chosen from disc stack centrifuges. In preferred examples of disc stack centrifuges, the feed enters under pressure through a nozzle at the bottom of the centrifuge in the liquid phase already present in the centrifuge and is accelerated to rotor speed, thus minimizing shear. In this embodiment, the feed may enter the ‘housing’ of the centrifuge via an inlet at the top of the centrifuge with the proviso that the feed only enters ‘the working volume’ of the centrifuge, where it mixes with the liquid phase already present, through a nozzle at the bottom of the centrifuge. The wet solids are accelerated by forces between about 5 and 14 times the force of gravity. Continuous discharge of wet solids occurs through a cyclone, a continuous discharge through peripheral nozzles or through intermittent discharge through valves that are opened at the bottom of the centrifuge.

Suitable disc stack centrifuges are for example Alfa Laval Clara 200 and Alfa Laval Brew 250 disc stack separation systems.

In a preferred embodiment, step (c) is performed under an atmosphere of molecular nitrogen (N₂).

The centrifugation used in the process of the invention is characterized in that it is performed by n serial centrifugation steps. Accordingly, the treated permeate P_{c} obtained in step (c) is subjected to n serial centrifugation steps, wherein n is an integer ranging from 2 to 5, wherein each centrifugation step i, wherein i is an integer between 1 and n, results in a pellet fraction Xᵢ and a centrate Cᵢ, wherein the centrate Cₓ of centrifugation step x, x being an integer ranging from 1 to n−1, is subjected to centrifugation in centrifugation step x+1.

In a preferred embodiment, n is 4, more preferably 3, even more preferably 2.

As described hereinbefore, the plant material preferably comprises green leaves comprising more than 5 wt. % of dry matter, more preferably more than 10 wt. %, even more preferably more than 15 wt. % and most preferably more than 20 wt. % of dry matter. The current invention is particularly suitable for obtaining soluble functional plant protein from green leaves comprising high dry matter values since the separation of green protein from soluble functional plant protein becomes more challenging at high dry matter values. When the green leaves comprise these high levels of dry matter, there is a need for at least 2 serially connected centrifuges to obtain a soluble functional plant protein fraction with less than 0.5 wt. % of wet solids, preferably less than 0.4 wt. %, more preferably less than 0.3 wt. %, even more preferably less than 0.2 wt. % and most preferably less than 0.1 wt. % of wet solids. The inventors have established that with higher levels of wet solids in the centrate Cₙ, the microfiltration membranes will irreversibly clog during the separation process and/or need to be cleaned too often.

Industrial scale centrifuges remove at most about 95 wt. % of the wet solids from the feed. The inventors have established that at least two serial centrifugation steps are needed to reduce the solids content of the centrate Cₙ to a level that is sufficiently low for a subsequent microfiltration step. The more centrifuges are serially connected, the lower the solids content of the centrate Cₙ of the last centrifuge in series will be. The lower the wet solids content of the centrate of the last centrifuge in series, the less protein retention will occur during microfiltration.

The centrate Cₙ obtained in the last centrifugation step (n as defined hereinbefore) has a wet solids content of 0.5 wt. % or less, based on the total weight of the centrate Cₙ, preferably less than 0.4 wt. %, more preferably less than 0.3 wt. %, even more preferably less than 0.2 wt. % and most preferably less than 0.1 wt. %.

The fact that the centrifuges are serially connected means that there is no different process step performed in between the subsequent centrifuging stages.

The pellet fractions Xᵢ are fractions with a relatively high solids content. They contain, however, still juice with soluble functional protein. The pellet fractions Xᵢ can be recycled as such (partly or completely), for example to the coarse physical separation step in step (b) or to the first centrifugation step in step (d), or they can be subjected to mechanical pressing first.

In preferred embodiments, one or more pellet fractions Xᵢ obtained in step (d) are subjected to mechanical pressing resulting in one or more pressed pellet fractions Xᵢ′ and one or more aqueous streams F_{c,i}. The one or more aqueous streams F_{c,i} comprising soluble functional proteins can then be recycled (partly or completely) to the purification process, for example to the mild treatment step in step (c) or to the first centrifugation step in step (d).

### Step (e) Microfiltration

In step (e), centrate Cₙ obtained in centrifugation step n is subjected to microfiltration.

Microfiltration is a membrane separation process, driven by a pressure gradient over the membrane, in which the membrane fractionates dissolved and dispersed components of a liquid as a function of their (solvated) size and structure.

Microfiltration as used herein refers to filtration over a membrane with a pore size of between 0.1 and 10 μm.

In a preferred embodiment, the pore size of the microfiltration membrane is between 0.1 and 5 μm, more preferably between 0.1 and 2.5 μm, even more preferably between 0.1 and 1 μm, still more preferably between 0.1 and 0.5 μm.

In another preferred embodiment, the pore size of the microfiltration membrane is between 0.2 and 0.5 μm, more preferably between 0.2 and 0.45 μm, even more preferably between 0.22 and 0.45 μm, still more preferably between 0.22 and 0.4 μm, even more preferably between 0.22 and 0.35 μm, yet more preferably between 0.22 and 0.3 μm.

The pore size of the microfiltration membrane is preferably such that it retains plant material membranes, such as chloroplast or cell membranes, chlorophyll, tannin, viruses, bacteria and/or aggregates thereof, while allowing passage of soluble functional proteins.

The microfiltration step results in a retentate Rₑ and a permeate Pₑ. The retentate Rₑ is the fraction that does not pass the microfiltration membrane. Accordingly, this fraction comprises larger molecules, such as chloroplast or cell membranes, chlorophyll, tannin, viruses, bacteria and/or aggregates thereof. The permeate Pₑ is the fraction that passes the microfiltration membrane. This fraction comprises smaller molecules, such as the soluble proteins. As will be appreciated by those skilled in the art, physical separation processes such as microfiltration are mostly not perfect in the sense that they typically do not separate a mixture into pure constituents. Consequently, as an example, the wording ‘the permeate comprises smaller molecules, such as the soluble proteins’ means that this fraction is at least enriched in soluble protein as compared to the retentate. Similarly, the wording ‘the retentate comprises larger molecules, such as plant material membranes’ means that this fraction it at least enriched in plant material membranes as compared to the permeate.

As explained hereinbefore, the wet solids content of the centrate Cₙ obtained in centrifugation step n that is subjected to microfiltration is 0.5 wt. % or less, based on the total weight of the permeate Pₙ.

If the wet solids content is higher than 0.5 wt. %, the microfiltration step may be less effective or the microfiltration membrane may get blocked.

Microfiltration is preferably performed at a temperature between 4 and 30° C. As explained hereinbefore, the process according to the invention is a process aimed at keeping the extracted soluble functional protein in solution until the soluble functional protein is dried. Within this temperature range, the white or soluble functional proteins remain in solution.

In a preferred embodiment, the membranes applied in the microfiltration step are hydrophilic membranes. Examples of hydrophilic membranes include, but are not limited to polymeric membranes comprising natural polymers (e.g. wool, rubber (polyisoprene), cellulose or synthetic polymers such as polyamide, modified or polar-functionalized membranes, or non-polymeric membranes comprising metal, ceramics, carbon zeolites.

In preferred embodiments, microfiltration step (e) is performed as a semi-continuous process or as a continuous process. In another preferred embodiment, the membrane configuration is cross-flow. Cross-flow configuration means that the stream that is subjected to filtration tangentially flows across the surface of the membrane. The advantage of this type of filtration is that any filter cake deposited onto the membrane is substantially washed away during the filtration process, increasing the length of time that the filtration unit can be operational.

As will be appreciated by those skilled in the art, 2 or more, such as 3, 4, or 5 microfiltration units connected in parallel can be applied instead of a single microfiltration unit. Moreover, as will be appreciated by those skilled in the art, 2 or more, such as 3, 4, or 5 microfiltration units connected serially can be applied instead of a single microfiltration unit. The 2 or more microfiltration units connected serially can have different pore sizes within the ranges defined herein, with decreasing pore size for microfiltration units positioned downstream.

In a preferred embodiment, microfiltration step (e) is performed as a semi-continuous process or as a continuous process with a cross-flow membrane configuration, wherein centrate Cₙ obtained in centrifugation step n is supplied to the high pressure side of a microfiltration membrane where part of the water and the smaller molecules cross the membrane to the lower pressure side, and wherein the permeate Pₑ is subjected to further downstream purification.

In a very preferred embodiment, microfiltration step (e) is performed as a semi-continuous process or as a continuous process with a cross-flow membrane configuration, wherein centrate Cₙ obtained in centrifugation step n is supplied to a buffer vessel, a stream discharged from the buffer vessel is continuously pumped to the high pressure side of a microfiltration membrane where part of the water and the smaller molecules cross the membrane to the lower pressure side, and wherein part of the retentate Rₑ is continuously recycled to the buffer vessel. This process requires purging at least part of the retentate Rₑ to prevent accumulation of solids.

In another very preferred embodiment, the microfiltration step (e) is performed as a semi-continuous process or as a continuous process with a cross-flow membrane configuration, wherein centrate Cₙ obtained in centrifugation step n is supplied to a buffer vessel, a stream discharged from the buffer vessel is continuously pumped to and distributed across the high pressure sides of 2 or more microfiltration units connected in parallel wherein part of the combined retentates Rₑ of each microfiltration unit is continuously recycled to the buffer vessel and wherein the permeates of each microfiltration unit are combined. The combined permeates are referred to as Pₑ. This process requires purging at least part of the combined retentates Rₑ to prevent accumulation of.

In still another very preferred embodiment, the microfiltration step (e) is performed as a semi-continuous process or as a continuous process with a cross-flow membrane configuration, wherein centrate Cₙ obtained in centrifugation step n is supplied to a buffer vessel, a stream discharged from the buffer vessel is continuously pumped to the high pressure side of the microfiltration membrane of the first microfiltration unit of 2 or more serially connected microfiltration units where part of the water and the smaller molecules cross the membrane to the lower pressure side, and wherein part of the retentate Rₑ leaving the last microfiltration unit in series is continuously recycled to the buffer vessel. This process requires purging at least part of the retentate Rₑ leaving the last microfiltration unit in series to prevent accumulation of solids.

Step (e) of microfiltration generally serves as a pre-treatment step for ultrafiltration step (f). Without microfiltration, ultrafiltration may be less effective or the ultrafiltration membrane may even get blocked or clogged. Anyway, without microfiltration, hydrophobic column adsorption cannot be performed.

The retentate Rₑ contains soluble functional protein. The retentate Rₑ can be recycled as such (partly or completely), for example to the coarse physical separation step in step (b) or to the first centrifugation step in step (d), or it can be subjected to mechanical pressing first.

In preferred embodiments, retentate Rₑ obtained in step (e) is subjected to mechanical pressing resulting in a pellet fraction Rₑ′ and an aqueous stream Fₑ. This aqueous stream Fₑ comprising soluble functional proteins can then be recycled (partly or completely) to the purification process, for example to the mild treatment step in step (c) or to the first centrifugation step in step (d).

### Step (f) Ultrafiltration/Diafiltration

In step (f), the permeate Pₑ (or the combined permeates Pₑ) of microfiltration step (e) is subjected to ultrafiltration.

Ultrafiltration is a membrane separation process, driven by a pressure gradient over the membrane, wherein the membrane fractionates dissolved and dispersed components of a liquid as a function of their (solvated) size and structure.

The main purpose of the ultrafiltration step is to concentrate the permeate Pₑ of microfiltration step (e) and to remove certain low-molecular weight compounds, such as salts, sugars and polyphenols that will be part of the permeate of P_{f} of ultrafiltration step (f). The soluble functional protein does not pass the ultrafiltration membrane. Consequently, the retentate R_{f} of ultrafiltration step (f) is the stream that is further processed downstream to obtain the soluble functional protein isolate.

Ultrafiltration as used herein refers to filtration over a membrane with a pore size of 0.01-0.1 μm or over a membrane having a molecular size cut-off of between 5 and 150 kDa, preferably between 10 and 150 kDa, more preferably between 20 and 150 kDa, even more preferably between 25 and 150 kDa, still more preferably between 30 and 150 kDa, yet more preferably between 40 and 150 kDa, such as between 50 and 150 kDa, between 75 and 150 kDa and between 100 and 150 kDa.

Examples of membranes that can be applied in the ultrafiltration step are polysulfone membranes, polyethersulfon membranes, cellulose acetate membranes, modified or polar-functionalized membranes, and ceramic membranes.

In a preferred embodiment the membrane configuration is a flat sheet membrane, such as disc membranes, flat plate membranes or spiral wound membranes, or a tubular membrane, such as a multi-channel membrane, a hollow fiber membrane or a honey comb membrane.

In preferred embodiments, the ultrafiltration step is performed as a semi-continuous or as a continuous process. In a very preferred embodiment, the membrane configuration is cross-flow.

Ultrafiltration is preferably performed at a temperature between 4 and 30° C. As explained hereinbefore, the process according to the invention is a process aimed at keeping the extracted soluble functional protein in solution until the soluble functional protein is dried. Within this temperature range, the white or soluble functional proteins remain in solution.

As will be appreciated by those skilled in the art, 2 or more, such as 3, 4, or 5 ultrafiltration units connected in parallel can be applied instead of a single ultrafiltration unit. Moreover, as will be appreciated by those skilled in the art, 2 or more, such as 3, 4, or 5 ultrafiltration units connected serially can be applied instead of a single ultrafiltration unit.

In a very preferred embodiment, ultrafiltration step (f) is performed as a semi-continuous process or as a continuous process with a cross-flow membrane configuration, wherein permeate Pₑ is supplied to the high pressure side of an ultrafiltration membrane where part of the water and the smaller molecules, such as salts, sugars and polyphenols, cross the membrane to the lower pressure side to form the permeate P_{f}, and wherein the retentate R_{f} is subjected to further downstream purification.

In another very preferred embodiment, ultrafiltration step (f) is performed as a semi-continuous process or as a continuous process with a cross-flow membrane configuration, wherein permeate Pₑ is supplied to a buffer vessel, a stream discharged from the buffer vessel is continuously pumped to the high pressure side of an ultrafiltration membrane where part of the water and the smaller molecules, such as salts, sugars and polyphenols, cross the membrane to the lower pressure side to form the permeate P_{f}, and wherein part of the retentate R_{f} is continuously recycled to the buffer vessel. The remaining part of the retentate R_{f} is subjected to further downstream purification.

In another very preferred embodiment, ultrafiltration step (f) is performed as a semi-continuous process or as a continuous process with a cross-flow membrane configuration, wherein permeate Pₑ is supplied to a buffer vessel, a stream discharged from the buffer vessel is continuously pumped to and distributed across the high pressure sides of 2 or more ultrafiltration units connected in parallel where part of the water and the smaller molecules, such as salts, sugars and polyphenols, cross the membrane to the lower pressure side to form the permeate, wherein part of the combined retentates R_{f} of each ultrafiltration unit is continuously recycled to the buffer vessel and wherein the permeates of each ultrafiltration unit are combined. The combined permeates are referred to as P_{f}. The remaining part of the combined retentates R_{f} is subjected to further downstream purification.

In still another very preferred embodiment, ultrafiltration step (f) is performed as a semi-continuous process or as a continuous process with a cross-flow membrane configuration, wherein permeate Pₑ is supplied to a buffer vessel, a stream discharged from the buffer vessel is continuously pumped to the high pressure side of the ultrafiltration membrane of the first ultrafiltration unit of 2 of more serially connected ultrafiltration units where part of the water and the smaller molecules, such as salts, sugars and polyphenols, cross the membrane to the lower pressure side to form the permeate, wherein part of the retentate R_{f} leaving the last ultrafiltration unit in series is continuously recycled to the buffer vessel and wherein the permeates of each ultrafiltration unit are combined. The combined permeates are referred to as P_{f}. The remaining part of the retentate R_{f} is subjected to further downstream purification.

In preferred embodiments, ultrafiltration in step (f) is performed as diafiltration. Diafiltration is a special type of ultrafiltration wherein the retentate from the ultrafiltration step is recycled and diluted with water before re-subjecting it to ultrafiltration.

In a very preferred embodiment, ultrafiltration step (f) is continuously or semi-continuously performed as diafiltration with a cross-flow membrane configuration, wherein permeate Pₑ is supplied to a buffer vessel, a stream discharged from the buffer vessel is continuously pumped to the high pressure side of an ultrafiltration membrane where part of the water and the smaller molecules, such as salts, sugars and polyphenols, cross the membrane to the lower pressure side to form the permeate P_{f}, and wherein part of the retentate R_{f} is diluted with water and continuously recycled to the buffer vessel. The remaining part of the retentate R_{f}, not further diluted with water, is subjected to further downstream purification.

In another very preferred embodiment, ultrafiltration step (f) is continuously or semi-continuously performed as diafiltration with a cross-flow membrane configuration, wherein permeate Pₑ is supplied to a buffer vessel, a stream discharged from the buffer vessel is continuously pumped to and distributed across the high pressure sides of 2 or more ultrafiltration units connected in parallel where part of the water and the smaller molecules, such as salts, sugars and polyphenols, cross the membrane to the lower pressure side to form the permeate, wherein part of the combined retentates R_{f} of each ultrafiltration unit is diluted with water and continuously recycled to the buffer vessel and wherein the permeates of each ultrafiltration unit are combined. The combined permeates are referred to as P_{f}. The remaining part of the combined retentates R_{f}, not further diluted with water, is subjected to further downstream purification.

In still another very preferred embodiment, ultrafiltration step (f) is continuously or semi-continuously performed as diafiltration with a cross-flow membrane configuration, wherein permeate Pₑ is supplied to a buffer vessel, a stream discharged from the buffer vessel is continuously pumped to the high pressure side of the ultrafiltration membrane of the first ultrafiltration unit of 2 of more serially connected ultrafiltration units where part of the water and the smaller molecules, such as salts, sugars and polyphenols, cross the membrane to the lower pressure side to form the permeate, wherein part of the retentate R_{f} leaving the last ultrafiltration unit in series is diluted with water and continuously recycled to the buffer vessel and wherein the permeates of each ultrafiltration unit are combined. The combined permeates are referred to as P_{f}. The remaining part of the retentate R_{f}, not further diluted with water, is subjected to further downstream purification.

Preferably, the ultrafiltration step concentrates the retentate R_{f} to between 25 and 50 wt. % dry matter, based on the total weight of the retentate R_{f}, more preferably to between 26 and 45 wt. %, still more preferably to between 27 and 40 wt. %.

Increasing the dry solids content, or reducing the amount of water, facilitates reduced transport volumes between the location of protein concentrate production on the one hand and purification and drying on the other hand. Moreover, since the final product is a dried soluble plant protein, removing water as early in the process is efficient.

Preferably, the ultrafiltration step reduces the salt concentration in the retentate R_{f} to concentrations resulting in a conductivity of below 10 mS/cm, more preferably to below 5 mS/cm, even more preferably of below 2 mS/cm, as measured with a WTW™ Proline™ Cond 3110 conductivity meter.

Preferably, the ultrafiltration step reduces the concentration of soluble phenolic content in the retentate R_{f} to below 1 mg eq gallic acid/(100 mg dry weight), such as between 0.01 and 0.8 mg eq gallic acid/(100 mg dry weight), between 0.015 and 0.5 mg eq gallic acid/(100 mg dry weight) or between 0.02 and 0.4 mg eq gallic acid/(100 mg dry weight), as measured with Folin-Ciocalteu reagent, in accordance with the method disclosed in E. A. Ainsworth and K. M. Gillespie, Estimation of total phenolic content and other oxidation substrates in plant tissue using Folin-Ciocalteu reagent, Nature Protocols, 2(4) 2007, pp 875-877, incorporated herein by reference. As regard this method, further reference is made to V. L. Singleton et al., Methods in Enzymology, 299 1999, pp 152-178, and to A. Kiskini et al., J. Agric. Food Chem., 64 2016, pp 8305-8314, both incorporated herein by reference.

### Step (g) Purification Over a Packed Column

Although the step of ultrafiltration, optionally combined with diafiltration, already removes part of the polyphenols, further reduction of the amount of polyphenols is needed.

In step (g), the retentate R_{f} of ultrafiltration step (f) is further purified by hydrophobic column adsorption. In this hydrophobic column adsorption step, the concentration of residual polyphenols and residual chlorophyll is reduced with a concomitant reduction of off-odors and/or off-flavors

This hydrophobic column adsorption comprises the use of a column packed with a hydrophobic adsorptive resin. A very suitable resin for removal of the residual phenolic compounds, off-odor and/or off-flavors, and residual chlorophyll was found to be a non-ionic crosslinked aromatic or aliphatic polymer resin, preferably a non-ionic crosslinked polystyrene resin, even more preferably a macroreticular styrene-divinylbenzene copolymer matrix. Such resins are commercially available under the names of Amberlite™ XAD-2, Amberlite™ XAD-4 and Amberlite™ XAD-16, Amberlite™ XAD 16N, Amberlite™ XAD 1180N, and Amberlite™ XAD 1600N (Sigma, St Louis, USA). The highly porous aliphatic acrylic adsorbent resin Amberlite™ XAD 7HP or highly porous phenolic adsorbent resin Amberlite™ XAD 761 (both available form Sigma, St Louis, USA) are also suitable for removal of the phenolic compounds. Amberlite™ XAD-16 is highly preferred as it results in excellent and almost simultaneous (or single pass) removal of residual chlorophyll, phenolic compounds, and other off-odors or off-flavor-causing compounds from the retentate R_{f}.

Other suitable and exemplary materials with the function of hydrophobic adsorption are talc, hydrophobized calcium carbonate, hydrophobized bentonite, hydrophobized kaolinite, hydrophobized glass, or a mixture thereof. Many hydrophobic adsorptive materials are commercially available, such as Toyopearl® Butyl-650, Tenax® TA™, Phenyl Sepharose™ Butyl Sepharose™, SOURCE™ 15 ethyl and SOURCE™ 15 phenyl media and Carbograph ITD™.

Preferably, the hydrophobic adsorption is performed by a column packed with hydrophobic adsorptive material, such as in the form of a matrix or beads. Preferably, the hydrophobic adsorption is performed by using an non-ionic crosslinked polystyrene resin, most preferably Amberlite™ XAD 16 resin.

The reduction in the polyphenol-concentration during hydrophobic adsorption, as may for instance be determined by measuring and comparing the absorption spectrum of the juice at 280 nm before and after the step of hydrophobic adsorption, is preferably at least 80 wt. % of the concentration in the green juice (permeate P_{b}) obtained in step (b). Preferably, the reduction is at least 90 wt. %, more preferably at least 95 or 98 wt. % of the concentration in the green juice (permeate P_{b}) obtained in step (b).

As will be appreciated by those skilled in the art, the hydrophobic column adsorption results in a column permeate P_{g}, whereas the impurities adsorbed (retentate R_{g}) remain on the static phase of the hydrophobic adsorption column. Although the step of hydrophobic column adsorption can be performed semi-continuously for at least some time, the hydrophobic column needs to be regenerated at regular time intervals to remove the adsorbed impurities. Such regeneration is preferably accomplished by the use of an NaOH 2-4% aqueous solution or ethanol as a desorption eluent to elute adsorbed compounds from the column, preferably using an NaOH 2-4% aqueous solution.

### Step (h) Drying

In step (h) the column permeate P_{g} from the hydrophobic adsorption column is dried to obtain dried functional soluble protein isolate from plant material.

The dried functional soluble protein isolate from plant material obtained in step (h) preferably takes the form of a free-flowing powder.

In a preferred embodiment, the dried functional soluble protein isolate from plant material obtained in step (h) has a dry solids content of more than 95 wt,%, preferably of more than 98 wt. %.

Drying is preferably performed by lyophilisation or spray drying.

The dried functional soluble protein from plant material obtained in step (h) is substantially odorless, and is substantially free of chlorophyll and polyphenols.

In a preferred embodiment the dried functional soluble protein obtained in step (h) has a purity of at least 70%, more preferably at least 85%, even more preferably at least 90% as measured by Kjeldahl analysis using a Buchi KjelMaster K-375 with a nitrogen conversion factor of 6.25.

In another preferred embodiment the dried functional soluble protein obtained in step (h) has a RuBisCo content of at least 80 wt %, more preferably of at least 90% based on the total weight of the dried functional soluble protein, as measured with HPLC SEC.

### Recycle Streams

The process according to the invention is characterized in that it comprises one or more recycle streams.

When the plant material employed in mechanical disruption step (a) consists of the leaves of sugar beet, retentate R_{b} typically constitutes about 30 v/v % of the feed stream of plant material supplied to mechanical disruption step (a). This retentate R_{b} then typically contains about 20 wt. % of the soluble functional protein present in mush stream Mₐ. Retentate R_{b} contains water, but only the water contained in the pulp, i.e. no free water that could be used to extract further soluble functional protein. So, recycling at least part of retentate R_{b} directly to coarse physical separation step (b) is preferably combined with diluting retentate R_{b} with additional fresh water. Alternatively, and more preferably, at least part of retentate R_{b} can be recycled to step (b) combined with another aqueous recycle stream, such as for example at least part E of retentate Rₑ.

When the plant material employed in mechanical disruption step (a) consists of the leaves of sugar beet, pellet fraction X₁ obtained in the first centrifugation step) typically constitutes about 30 v/v % of the feed stream P_{c} supplied to the first centrifuge. This pellet fraction X₁ then typically contains about 10-30 wt. %, such as 20 wt/%, of the soluble functional protein present in the feed P_{c}. Although pellet fraction X₁ contains some water, directly recycling at least part of pellet fraction X₁ to the first centrifugation step of step (d) or to the mild treatment step of step (c) will adversely affect the separation since the resulting solids content of combined streams X₁ and P_{c} will be too high. Preferably, at least part of pellet fraction X₁ is diluted with another aqueous recycle stream, such as for example at least part E of retentate Rₑ.

Alternatively, pellet fraction X₁ can be subjected to mechanical pressing resulting in a pressed pellet fraction Xᵢ′ and an aqueous stream F_{c,1}, such that at least part of the aqueous stream F_{c,1} can be recycled to the first centrifugation step of step (d) or to the mild treatment step of step (c).

When the plant material employed in mechanical disruption step (a) consists of the leaves of sugar beet, pellet fraction X₂ obtained in the second centrifugation step in series typically constitutes about 5 v/v % of the feed stream P_{c} This pellet fraction X₂ then typically contains about 3-10 wt. %, such as 5 wt. %, of the soluble functional protein present in the feed P_{c}. Pellet fraction X₂ contains a substantial amount of water such that (at least part of it) it can be recycled, eventually in combination with (at least part of) pellet fraction X₁ to the first centrifugation step in step (d).

When the plant material employed in mechanical disruption step (a) consists of the leaves of sugar beet, retentate Rₑ typically constitutes less than about 5 v/v % of the feed stream Pₑ. This retentate Rₑ then typically contains about 10-30 wt. %, such as 20 wt. %, of the soluble functional protein present in the feed Pₑ. At least part of this stream can be recycled along with other streams, mainly to add water to the other recycle streams.

Accordingly, the method comprises a recycling step comprising:
(AA) recycling at least part B of retentate R_{b}, at least part Dᵢ of stream Xᵢ, wherein i is an integer selected from 1 to n, at least part E of retentate Rₑ, or combinations thereof, to the coarse physical separation step in step (b);
(BB) when mechanical pressing is performed in any one of steps (b), (d) or (e), recycling at least part B′ of aqueous stream F_{b}, at least part Dᵢ′ of aqueous stream F_{c,i}, wherein i is an integer selected from 1 to n, at least part E′ of aqueous stream Fₑ, or combinations thereof, to the mild treatment step in step (c);
(CC) recycling at least part Dᵢ of stream Xᵢ, wherein i is an integer selected from 1 to n, at least part E of retentate Rₑ, or combinations thereof, to the first centrifugation step in step (d); or
(DD) when mechanical pressing is performed in any one of steps (b), (d) or (e), recycling at least part Dᵢ of stream Xᵢ, at least part Dᵢ′ of aqueous stream F_{c,i}, wherein i is an integer selected from 1 to n, at least part E of retentate Rₑ, at least part E′ of aqueous stream Fₑ, or combinations thereof, to the first centrifugation step in step (d).

Where possible, recycle steps (AA)-(DD) can be combined.

According to the common understanding of the skilled person, the wording ‘recycling at least part of stream S, at least part of stream T, or combinations thereof’ means that at least part of (including all of) the individual stream S, or at least part of (including all of) the individual stream T, or any combination of at least part of (including all of) the individual streams S and T can be recycled. Moreover, according to the common understanding of the skilled person, the wording ‘or at least part Dᵢ of stream Xᵢ, wherein i is an integer selected from 1 to n’ means ‘or at least part D₁ of stream Xᵢ, or . . . , or at least part Dₙ of stream Xₙ’.

Accordingly, the recycling steps in the method as defined herein can also be worded as:
(AA) recycling at least part B of retentate R_{b}, or at least part Dᵢ of stream Xᵢ, wherein i is an integer selected from 1 to n, or at least part E of retentate Rₑ, or combinations thereof, to the coarse physical separation step in step (b);
(BB) when mechanical pressing is performed in any one of steps (b), (d) or (e), recycling at least part B′ of aqueous stream F_{b}, or at least part Dᵢ′ of aqueous stream F_{c,i}, wherein i is an integer selected from 1 to n, or at least part E′ of aqueous stream Fₑ, or combinations thereof, to the mild treatment step in step (c);
(CC) recycling at least part Dᵢ of stream Xᵢ, wherein i is an integer selected from 1 to n, or at least part E of retentate Rₑ, or combinations thereof, to the first centrifugation step in step (d); or
(DD) when mechanical pressing is performed in any one of steps (b), (d) or (e), recycling at least part Dᵢ of stream Xᵢ, or at least part Dᵢ′ of aqueous stream F_{c,i}, wherein i is an integer selected from 1 to n, or at least part E of retentate Rₑ, or at least part E′ of aqueous stream Fₑ, or combinations thereof, to the first centrifugation step in step (d).

As will be appreciated by the skilled person, only streams can be recycled that have been made available in steps (b), (d) and (e). So, if for example mechanical pressing is performed in step (b), then aqueous stream F_{b} is provided instead of retentate R_{b}, meaning that only at least part B′ of aqueous stream F_{b} can be recycled. Likewise, if mechanical pressing is performed for example in the j^{th} centrifugation step in step (d) only and not in centrifugation steps i 1 j, wherein i is an integer selected from 1 to n, then at least part Dⱼ′ of aqueous stream F_{c}j can be recycled, or at least part Dᵢ of stream Xᵢ, or combinations thereof.

Accordingly, some recycling steps corresponding to (BB) in the method as defined herein can also be worded as:
when mechanical pressing is performed in step (b), recycling at least part B′ of aqueous stream F_{b}, to the mild treatment step in step (c);
when mechanical pressing is performed in one or more centrifugation steps i in step (d), recycling at least part Dᵢ′ of aqueous stream F_{c,i}, wherein i is an integer selected from 1 to n, to the mild treatment step in step (c);
when mechanical pressing is performed in step (e), recycling at least part E′ of aqueous stream Fₑ, or combinations thereof, to the mild treatment step in step (c);
when mechanical pressing is performed in step (b) and in one or more centrifugation steps i in step (d), recycling at least part B′ of aqueous stream F_{b}, or at least part Dᵢ′ of aqueous stream F_{c,i}, wherein i is an integer selected from 1 to n, or combinations thereof, to the mild treatment step in step (c);
when mechanical pressing is performed in steps (b) and (e), recycling at least part B′ of aqueous stream F_{b}, or at least part E′ of aqueous stream Fₑ, or combinations thereof, to the mild treatment step in step (c);
when mechanical pressing is performed in step (e) and in one or more centrifugation steps i in step (d), recycling at least part Dᵢ′ of aqueous stream F_{c,i}, wherein i is an integer selected from 1 to n, or at least part E′ of aqueous stream Fₑ, or combinations thereof, to the mild treatment step in step (c); and
when mechanical pressing is performed in step (b), in one or more centrifugation steps i in step (d) and in step (e), recycling at least part B′ of aqueous stream F_{b}, or at least part Dᵢ′ of aqueous stream F_{c,i}, wherein i is an integer selected from 1 to n, or at least part E′ of aqueous stream Fₑ, or combinations thereof, to the mild treatment step in step (c).

Some recycling steps corresponding to (DD) in the method as defined herein can also be worded as:
when mechanical pressing is performed in one or more centrifugation steps i in step (d), wherein said one or more steps i are chosen from integers from 1 to n, recycling at least part Dᵢ′ of aqueous stream F_{c,i}, or at least part Dⱼ of stream Xⱼ, wherein j is an integer selected from 1 to n and wherein j≠i, or at least part E of retentate Rₑ, or combinations thereof, to the first centrifugation step in step (d); and
when mechanical pressing is performed in step (e) and in one or more centrifugation steps i in step (d), wherein said one or more steps i are chosen from integers from 1 to n, recycling at least part Dᵢ′ of aqueous stream F_{c,i}, or at least part Dⱼ of stream Xⱼ, wherein j is an integer selected from 1 to n and wherein j≠i, or at least part E′ of aqueous stream Fₑ, or combinations thereof, to the first centrifugation step in step (d).

Thus, the invention has been described by reference to certain embodiments discussed above. It will be recognized that these embodiments are susceptible to various modifications and alternative forms well known to those of skill in the art.

Furthermore, for a proper understanding of this document and its claims, it is to be understood that the verb ‘to comprise’ and its conjugations are used in its non-limiting sense to mean that items following the word are included, but items not specifically mentioned are not excluded. In addition, reference to an element by the indefinite article ‘a’ or ‘an’ does not exclude the possibility that more than one of the element is present, unless the context clearly requires that there be one and only one of the elements. The indefinite article ‘a’ or ‘an’ thus usually means ‘at least one’.

### EXAMPLES

In the following examples, methods for obtaining soluble functional protein isolate from sugar beet leaves are illustrated. The effect of different recycle steps on the resulting overall yield of the soluble functional protein isolate is demonstrated.

The general experimental setup comprises collecting and crushing the sugar beet leaves (mechanical disruption step (a)) in a mill from Bruckner Liquid Food Tech GmbH, followed by extraction and separation of the juice (coarse physical separation step (b)) in an UDE screw press from Bruckner Liquid Food Tech GmbH. In this UDE screw press, a screw pile with 2 helical threads and a pitch size of between 5 and 20 cm was used at a rotation speed of 60 Hz. The distance between the screw pile and filter unit was 12 mm. The filter had pores with a diameter of 500 m. The pressure applied on the permeate-side of the filter element was 200 mbar, resulting in a pressure difference across the filter element of about 800 mbar. The height of the helical threads (the difference between the diameter of the helical threads and the shaft diameter) was 12 mm. Between coarse physical separation step (b) and mild treatment (step (c)), 10 liter of an 20% aqueous sodium bisulfite solution was added to 1000 liter of the extracted and separated juice. Subsequently, before mild treatments step (c), 50 liter of an 35% aqueous CaCl₂ solution was added.

The extracted and separated juice was subjected to mild treatment (step (c)) by heating to 47° C. for 5 min using a XLG® heat exchanger. Next, the treated juice was centrifuged (step (d)) using two serial disc stack centrifuges, Clara 200 and Brew 250 (both Alfa Laval). The centrate of the second centrifuge was subjected to microfiltration (Alva Laval) using a ceramic membrane with a pore size of 0.45 m (step (e)). The resulting protein solution was purified in an ultra- and diafiltration process (step (f)) using a spiral wound membrane with a pore size of 100 kD (Sartorius). In a subsequent step, the retentate of the ultra- and diafiltration process was subjected to purification in a column (step (g)) packed with Amberlite FPX 66. Finally, the resulting column permeate was dried (step (h)) to afford the soluble functional protein isolate.

### Comparative Example 1

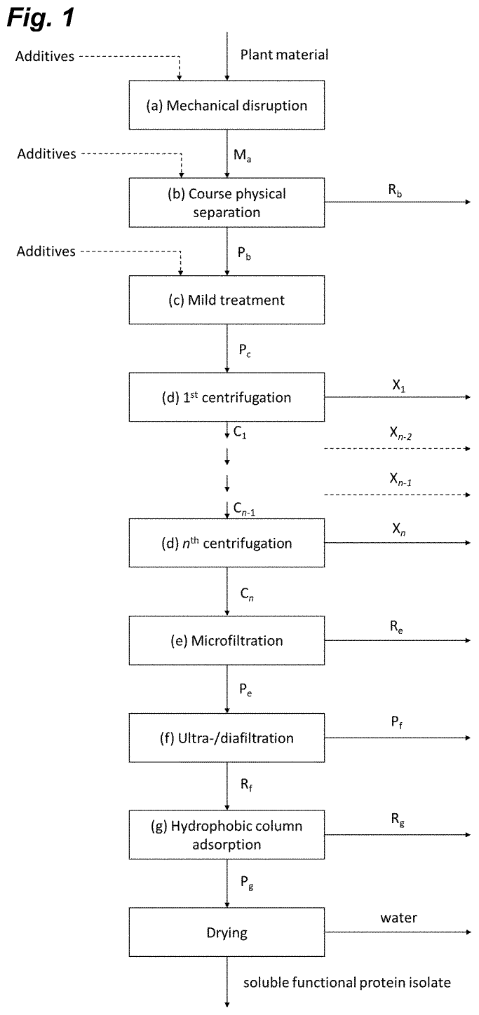

In a comparative example, no recycling steps were performed (see FIG. 1 with n=2). This comparative example is based on 62% juice yield in the coarse physical separation step, and a concentration factor of CF10 between the microfiltration and dia-/ultrafiltration step. The soluble functional protein isolate was afforded in a 27% yield, based on the mass of the soluble functional protein present in the mash Mₐ (see Table 2).

### Example 2

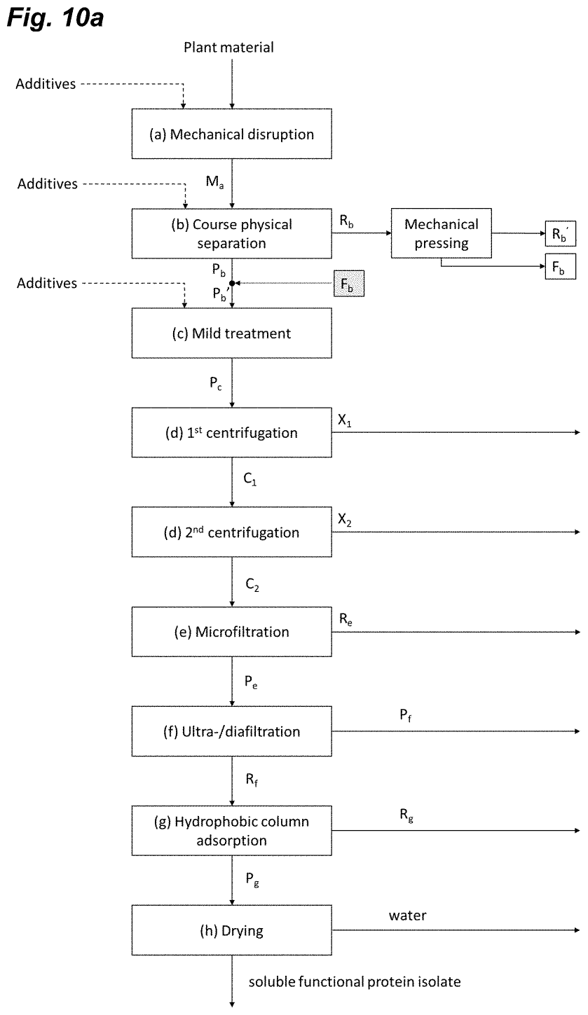

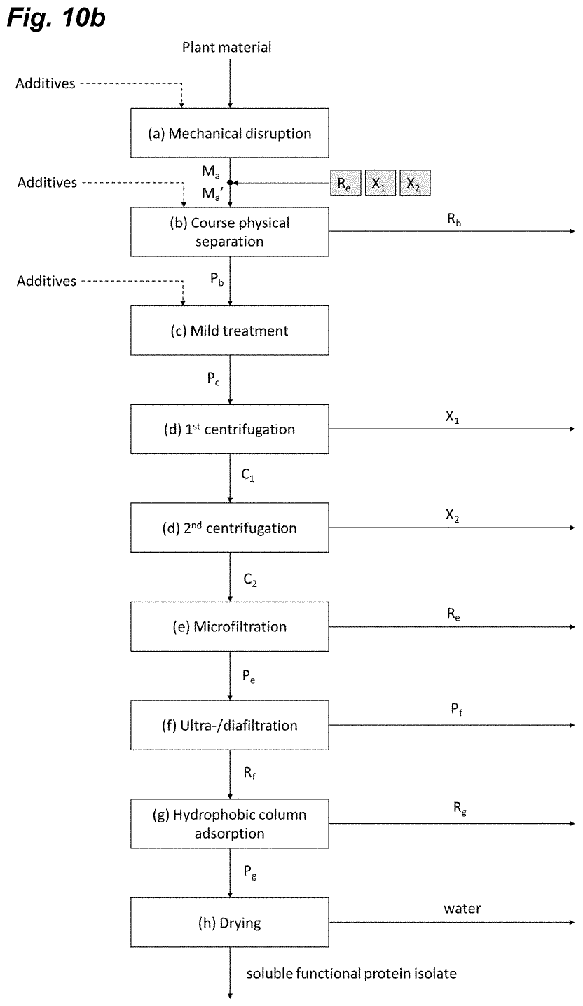

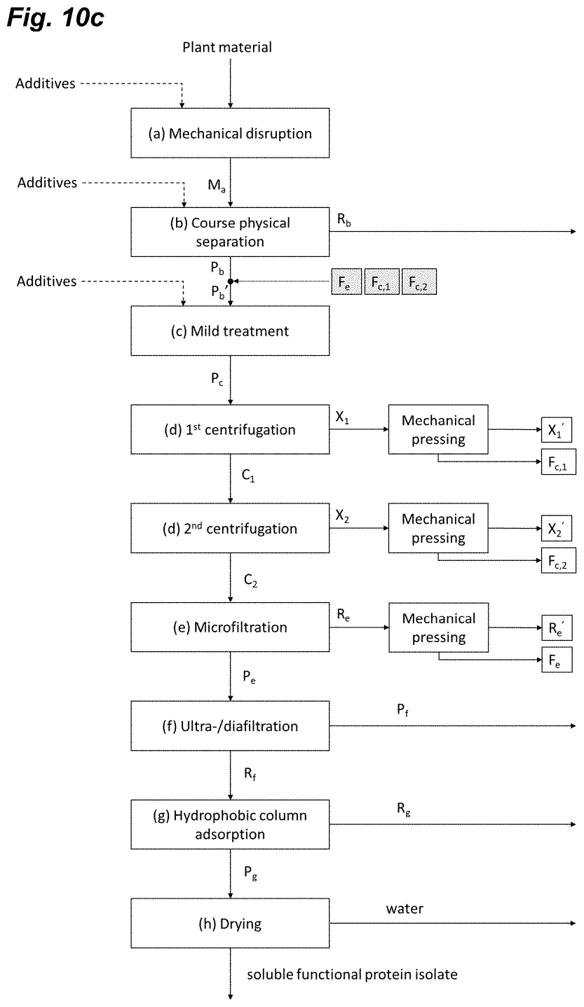

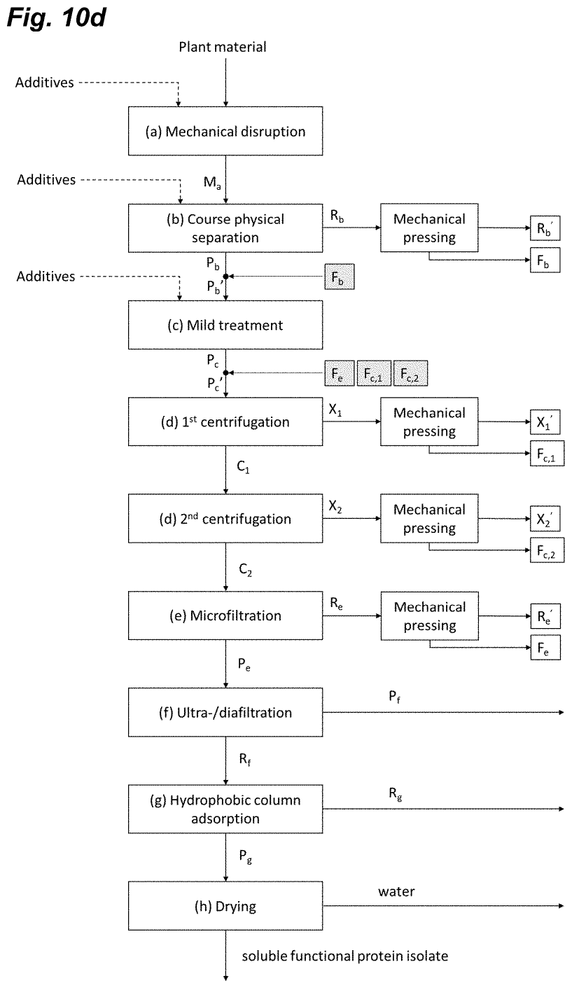

In this example, the retentate R_{b} obtained in coarse physical separation step (b) was further subjected to mechanical pressing in coarse physical separation step (b) in a screw press from Babbini, resulting in a concentrated fibre stream R_{b}′ and an aqueous stream F_{b}. The whole aqueous stream F_{b} was recycled to the mild treatment step (c). See FIG. 10a. The soluble functional protein isolate was afforded in a 39% yield, based on the mass of the soluble functional protein present in the mash Mₐ (see Table 3).

### Example 3

In this example, the pellet fractions X₁ and X₂ obtained in centrifugation step (d) and the retentate Rₑ obtained in microfiltration step (e) were recycled as a whole to the coarse physical separation step (b). See FIG. 10b. The soluble functional protein isolate was afforded in a 39% yield, based on the mass of the soluble functional protein present in the mash Mₐ (see Table 4).

### Example 4

In this example, the pellet fractions X₁ and X₂ obtained in centrifugation step (d) were further subjected in centrifugation step (d) to mechanical pressing in a filter press from Andritz resulting in pressed pellet fractions Xᵢ′ and X₂′ and aqueous streams F_{c,1} and F_{c,2}. Moreover, retentate Rₑ obtained in microfiltration step (e) was further subjected in microfiltration step (e) to mechanical pressing in a filter press from Andritz, resulting in a pellet fraction Rₑ′ and an aqueous stream Fₑ. The whole aqueous streams F_{c,1}, F_{c,2} and Fₑ were recycled to the mild treatment step (c). See FIG. 10c. The soluble functional protein isolate was afforded in a 43% yield, based on the mass of the soluble functional protein present in the mash Mₐ (see Table 5).

### Example 5

In this example, the retentate R_{b} obtained in coarse physical separation step (b) was further subjected to mechanical pressing in coarse physical separation step (b) in a screw press from Babbini, resulting in a concentrated fibre stream R_{b}′ and an aqueous stream F_{b}. Furthermore, the pellet fractions X₁ and X₂ obtained in centrifugation step (d) were further subjected in centrifugation step (d) to mechanical pressing in a filter press from Andritz, resulting in pressed pellet fractions Xᵢ′ and X₂′ and aqueous streams F_{c,1} and F_{c,2}. Moreover, retentate Rₑ obtained in microfiltration step (e) was further subjected in microfiltration step (e) to mechanical pressing in a filter press from Andritz, resulting in a pellet fraction Rₑ′ and an aqueous stream Fₑ. The whole aqueous stream F_{b} was recycled to the mild treatment step (c). The whole aqueous streams F_{c,1}, F_{c,2} and Fₑ were recycled to the first centrifugation step in step (d). See FIG. 10d. The soluble functional protein isolate was afforded in a 53% yield, based on the mass of the soluble functional protein present in the mash Mₐ (see Table 6).

### Example 6

In this example, the effect of the type of centrifuge applied in step (d) of the process as claimed on the turbidity and/or the particle size distribution of the centrate is investigated.

In a first test, steps (a)-(c) of the process as claimed were performed on endive leaves as starting material to provide a treated permeate P_{c}. In the first test, treated permeate P_{c} comprising aggregates or flocculates obtained in mild treatment step (c) was subjected to centrifuging in a GEA decanter centrifuge (speed: 6769 rpm, differential speed: 10 rpm, fluid level: 23 mm) and a disc stack centrifuge (Gea Westfalia SSD-2, speed: 11.000 rpm) in series. The decanter centrifuge separated the crude solids (pellet fraction X₁) from a ‘deca-juice’ (centrate C₁). The deca-juice was fed to the disc stack centrifuge and the fine solids (pellet fraction X₂) were separated from a ‘centri-juice’ (centrate C₂).

The first test showed that the decanter centrifuge not only separated off part of the crude solids, but also disintegrated the aggregates or flocculates formed during mild treatment step (c). As a result of this disintegration, the suspended solids became smaller. The disintegration of the aggregates or flocculates to smaller particles, more particularly to disintegrated membrane fragments, manifested itself in an increased turbidity of Centrate C₁ as compared to treated permeate P_{c}. As a result, it was more difficult to remove the disintegrated membrane fragments in the disc stack centrifuge connected in series, which causes difficulties in the downstream process.

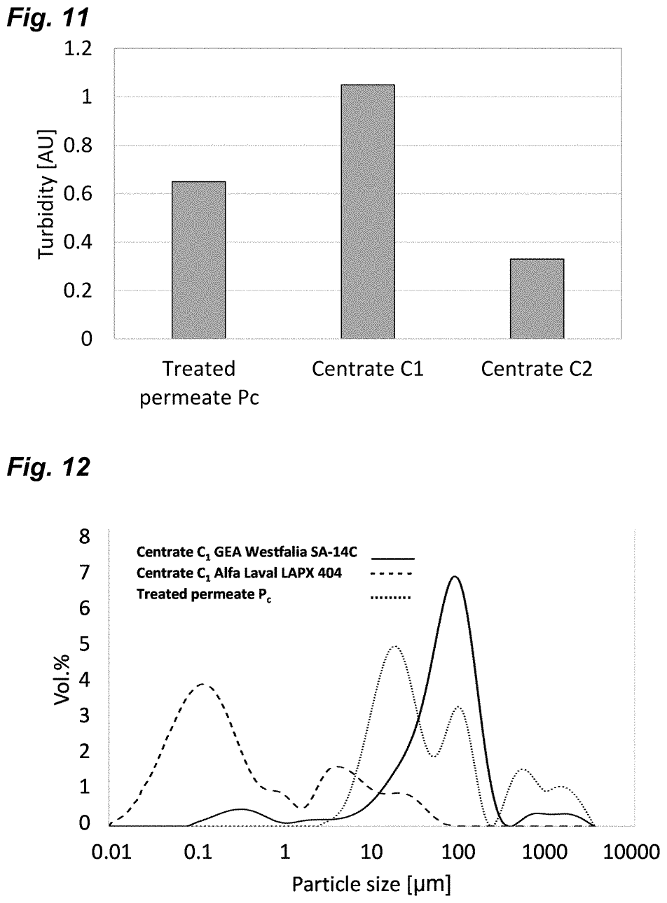

FIG. 11 shows the turbidity (measured as a relative value, in absorption units (AU) at 800 nm on undiluted samples) of the treated permeate P_{c}, i.e. after mild treatment step (c), of the centrate C₁ after the first centrifuging step (d) with the decanter centrifuge and of the centrate C₂ after the second centrifuging step (d) with the disc stack centrifuge. A decrease in turbidity after the decanter centrifuging step would have been expected. Nevertheless, the turbidity increased, indicating that more individual solid particles (disintegrated membrane fragments) are present.

In a second test, steps (a)-(c) of the process as claimed were performed on a mixture of endive and lettuce leaves as starting material to provide a treated permeate P_{c}. In the second test, the effect of single centrifuge configurations was tested, i.e. treated permeate P_{c} was only processed over one centrifuge, to determine the exact effect of the centrifuge configuration on the centrate quality. Two different centrifuges were tested: the GEA Westfalia SA-14 and the Alfa Laval LAPX 404. The GEA Westfalia SA-14, that was operated at 12000 rpm, is a high impact centrifuge, wherein the feed enters at the top of the centrifuge directly onto the discs. The Alfa Laval LAPX 404 is a demo model centrifuge wherein the feed enters at the top of the centrifuge and wherein the rotation per minute (rpm) can be varied. The Alfa Laval LAPX 404 was operated at 6500 rpm, thereby mimicking low impact centrifuges available at Alfa Laval, like Alfa Laval Clara 200 wherein the feed enters under pressure through a nozzle at the bottom of the centrifuge in the liquid phase already present in the centrifuge and is accelerated to rotor speed.

The (volume-based) particle size distribution in the treated permeate P_{c} and in the centrates C₁ obtained after centrifugation in the second test was determined using a Mastersizer 3000 (FIG. 12). The treated permeate P_{c} processed on the GEA Westfalia SA-14 had a high turbidity. The part particle size distribution results show that the GEA Westfalia SA-14 only limitedly removed large particles. The increase in turbidity, together, with the limited removal of particles over 1 m made the inventors conclude that the Gea Westfalia SA-14 high impact centrifuge at least partly disintegrated the particles formed during the heat coagulation step (c) in a similar manner as a decanter centrifuge. The treated permeate P_{c} processed on the Alfa Laval LAPX 404 had a low turbidity. The particle size distribution results show that the Alfa Laval LAPX 404 removed large particles and did not disintegrate the aggregates or flocculates obtained in mild treatment step (c).

TABLE 2

| | | | Sol. Func. | Sol. Func. | Yield(/loss) |
|---|---|---|---|---|---|
| | Indication | Mass | Protein | Protein | [%] Mash |
| Process stream | in FIG. 1 | [kg] | [%] | [kg] | as 100% |
| Coarse physical separation | | | | | |
| Mash | Ma | 1622 | | 17.74 | 100 |
| Fibres | Rb | 622 | | | |
| Juice | Pb | 1000 | 1.1 | 11.00 | 62 |
| 1st centrifugation | | | | | |
| Juice | Pb | 1000 | 1.10 | 11.00 | 62 |
| Centrate 1 | C1 | 855 | 0.83 | 7.05 | 40 |
| Pellet 1 | X1 | 145 | | 3.95 | 22 |
| 2nd centrifugation | | | | | |
| Centrate 1 | C1 | 855 | 0.83 | 7.05 | 40 |
| Centrate 2 | C2 | 836 | 0.83 | 6.90 | 39 |
| Pellet 2 | X2 | 19 | | 0.16 | 1 |
| Microfiltration | | | | | |
| Centri-juice 2 | C2 | 836 | 0.83 | 6.90 | 39 |
| MF retentate | Re | 100 | 1.72 | 1.72 | 10 |
| MF permeate | Pe | 736 | 0.70 | 5.17 | 29 |
| Ultrafiltration | | | | | |
| MF permeate | Pe | 736 | 0.70 | 5.17 | 29 |
| UF permeate | Pf | 662 | 0.05 | 0.33 | 2 |
| UF retentate | Rf | 74 | 6.58 | 4.84 | 27 |

TABLE 3

| | | | Sol. Func. | Sol. Func. | Yield(/loss) |
|---|---|---|---|---|---|
| | Indication | Mass | Protein | Protein | [%] Mash |
| Process stream | in FIG. 10a | [kg] | [%] | [kg] | as 100% |
| Coarse physical separation | | | | | |
| Mash | Ma | 1622 | | 17.74 | 100 |
| Fibres | Rb | 622 | | | |
| Juice | Pb | 1000 | 1.10 | 11.00 | 62 |
| Mechanical pressing | | | | | |
| Fibers | Rb | 622 | | | 0 |
| Pressed fibres | Rb′ | 412 | | | 0 |
| Pressed juice recycle | | | | | |
| stream | Fb | 210 | 1.10 | 2.31 | 13 |
| Mild treatment | | | | | |
| Juice | Pb′ = Pb + Fb | 1210 | 1.10 | 13.31 | 75 |
| Treated juice | Pc | 1210 | 1.10 | 13.31 | 75 |
| 1st centrifugation | | | | | |
| Treated juice | Pc | 1210 | 1.10 | 13.31 | 75 |
| Centrate 1 | C1 | 1065 | 0.94 | 9.96 | 56 |
| Pellet 1 | X1 | 145 | | 3.35 | 19 |
| 2nd centrifugation | | | | | |
| Centrate 1 | C1 | 1065 | 0.94 | 9.96 | 56 |
| Centrate 2 | C2 | 1046 | 0.94 | 9.78 | 55 |
| Pellet 2 | X2 | 19 | | 0.18 | 1 |
| Microfiltration | | | | | |
| Centri-juice 2 | C2 | 1046 | 0.94 | 9.78 | 55 |
| MF retentate | Re | 100 | 2.44 | 2.44 | 14 |
| MF permeate | Pe | 946 | 0.78 | 7.33 | 41 |
| Ultrafiltration | | | | | |
| MF permeate | Pe | 946 | 0.78 | 7.33 | 41 |
| UF permeate | Pf | 851 | 0.05 | 0.43 | 2 |
| UF retentate | Rf | 95 | 7.30 | 6.91 | 39 |

TABLE 4

| | | | Sol. Func. | Sol. Func. | Yield(/loss) |
|---|---|---|---|---|---|
| | Indication | Mass | Protein | Protein | [%] Mash |
| Process stream | in FIG. 10b | [kg] | [%] | [kg] | as 100% |
| Coarse physical separation | | | | | |
| Mash | Ma | 1622 | | 17.74 | 100 |
| Fibres | Rb (1) | 622 | | | |
| Juice | Pb (1) | 1000 | 1.10 | 11.00 | 62 |
| Recycle streams: | | | | | |
| Coarse physical separation | | | | | |
| Recycle streams | X1 + X2 + Re | | 264 | | 0 |
| Fibres | Rb (2) | 100 | | | 0 |
| Juice | Pb (2) | 164 | 1.55 | 2.53 | 14 |
| Mild treatment | | | | | |
| Juice | Pb′ = Pb (1) + Pb (2) | 1164 | 1.16 | 13.53 | 76 |
| Treated juice | Pc | 1164 | 1.16 | 13.53 | 76 |
| 1st centrifugation | | | | | |
| Juice | Pc | 1164 | 1.16 | 13.53 | 76 |
| Centrate 1 | C1 | 1019 | 0.99 | 10.04 | 57 |
| Pellet 1 | X1 | 145 | | 3.49 | 20 |
| 2nd centrifugation | | | | | |
| Centrate 1 | C1 | 1019 | 0.99 | 10.04 | 57 |
| Centrate 2 | C2 | 1000 | 0.99 | 9.86 | 56 |
| Pellet 2 | X2 | 19 | | 0.19 | 1 |
| Microfiltration | | | | | |
| Centrate 2 | C2 | 1000 | 0.99 | 9.86 | 56 |
| MF retentate | Re | 100 | 2.46 | 2.46 | 14 |
| MF permeate | Pe | 900 | 0.82 | 7.39 | 42 |
| Ultrafiltration | | | | | |
| MF permeate | Pe | 900 | 0.82 | 7.39 | 42 |
| UF permeate | Pf | 810 | 0.05 | 0.40 | 2 |
| UF retentate | Rf | 90 | 7.77 | 6.99 | 39 |

TABLE 5

| | | | Sol. Func. | Sol. Func. | Yield(/loss) |
|---|---|---|---|---|---|
| | Indication | Mass | Protein | Protein | [%] Mash |
| Process stream | in FIG. 10c | [kg] | [%] | [kg] | as 100% |
| Coarse physical separation | | | | | |
| Mash | Ma | 1622 | | 17.74 | 100 |
| Fibers | Rb | 622 | | | |
| Juice | Pb | 1000 | 1.10 | 11.00 | 62 |
| Recycle streams: | | | | | |
| Mechanical pressing | | | | | |
| Streams to mechanical pressing | X1 + X2 + Re | 264 | | | 0 |
| Fiber from mechanical pressing | X1′ + X2′ + Re′ | 40 | | | 0 |
| Recycle stream | Fc, 1 + Fc, 2 + Fe | 224 | 1.64 | 3.69 | 21 |
| Mild treatment | | | | | |
| Juice | Pb′ = Pb + Fc, 1 + Fc, 2 + Fe | 1224 | 1.20 | 14.69 | 83 |
| Treated juice | Pc | 1224 | 1.20 | 14.69 | 83 |
| 1st centrifugation | | | | | |
| Juice | Pc | 1224 | 1.20 | 14.69 | 83 |
| Centrate 1 | C1 | 1079 | 1.01 | 10.92 | 62 |
| Pellet 1 | X1 | 145 | | 3.77 | 21 |
| 2nd centrifugation | | | | | |
| Centrate 1 | C1 | 1079 | 1.01 | 10.92 | 62 |
| Centrate 2 | C2 | 1060 | 1.01 | 10.73 | 60 |
| Pellet 2 | X2 | 19 | | 0.19 | 1 |
| Microfiltration | | | | | |
| Centrate 2 | C2 | 1060 | 1.01 | 10.73 | 60 |
| MF retentate | Re | 100 | 2.68 | 2.68 | 15 |
| MF permeate | Pe | 960 | 0.84 | 8.04 | 45 |
| Ultrafiltration | | | | | |
| MF permeate | Pe | 960 | 0.84 | 8.04 | 45 |
| UF permeate | Pf | 864 | 0.05 | 0.43 | 2 |
| UF retentate | Rf | 96 | 7.93 | 7.61 | 43 |

TABLE 6

| | | | Sol. Func. | Sol. Func. | Yield(/loss) |
|---|---|---|---|---|---|
| | Indication | Mass | Protein | Protein | [%] Mash |
| Process stream | in FIG. 10d | [kg] | [%] | [kg] | as 100% |
| Coarse physical separation | | | | | |
| Mash | Ma | 1622 | | 17.74 | 100 |
| Fibers | Rb | 622 | | | |
| Juice | Pb | 1000 | 1.10 | 11.00 | 62 |
| Recycle streams: | | | | | |
| Mechanical pressing | | | | | |
| Fibers | Rb | 622 | | | 0 |
| Pressed fibers | Rb′ | 412 | | | 0 |
| Pressed juice | Fb | 210 | 1.10 | 2.31 | 13 |
| Recycle streams: | | | | | |
| Mechanical pressing | | | | | |
| Streams to mechanical pressing | X1 + X2 + Re | 264 | | | 0 |
| Fiber from mechanical pressing | X1′ + X2′ + Re′ | 40 | | | 0 |
| Recycle stream | Fc, 1 + Fc, 2 + Fe | 224 | 1.89 | 4.24 | 24 |
| Mild treatment | | | | | |
| Juice | Pb′ = Pb + Fb | | | | |
| Treated juice | Pc | | | | |
| 1st centrifugation | | | | | |
| Juice | Pc′ = Pc + Fc, 1 + Fc, 2 + Fe | 1434 | 1.22 | 17.55 | 99 |
| Centrate 1 | C1 | 1289 | 1.04 | 13.37 | 75 |
| Pellet 1 | X1 | 145 | | 4.18 | 24 |
| 2nd centrifugation | | | | | |
| Centrate 1 | C1 | 1289 | 1.04 | 13.37 | 75 |
| Centrate 2 | C2 | 1270 | 1.04 | 13.17 | 74 |
| Pellet 2 | X2 | 19 | | 0.20 | 1 |
| Microfiltration | | | | | |
| Centrate 2 | C2 | 1270 | 1.04 | 13.17 | 74 |
| MF retentate | Re | 100 | 3.29 | 3.29 | 19 |
| MF permeate | Pe | 1170 | 0.84 | 9.88 | 56 |
| Ultrafiltration | | | | | |
| MF permeate | Pe | 1170 | 0.84 | 9.88 | 56 |
| UF permeate | Pf | 1053 | 0.05 | 0.53 | 3 |
| UF retentate | Rf | 117 | 7.99 | 9.35 | 53 |

1. A method for isolating soluble functional plant protein from a plant material comprising the following steps:
a) mechanically disrupting the cells of the plant material to obtain a mush stream Mₐ comprising plant juice and disrupted cells;
b) subjecting the mush stream Mₐ obtained in step (a) to a coarse physical separation step wherein the plant juice is separated from a pulp comprising disrupted cells, resulting in a permeate P_{b} comprising plant juice and a retentate R_{b} comprising disrupted cells, wherein the retentate R_{b} is optionally subjected to mechanical pressing resulting in a concentrated fibre stream R_{b}′ and an aqueous stream F_{b};
c) subjecting the permeate P_{b} obtained in step (b) to mild treatment at a temperature between 20° C. and 60° C. for at least 1 minute, optionally in the presence of one or more flocculants, resulting in a treated permeate P_{c} comprising aggregates or flocculates;
d) subjecting the treated permeate P_{c} obtained in step (c) to n serial centrifugation steps, wherein n is an integer ranging from 2 to 5, wherein each centrifugation step i, wherein i is an integer between 1 and n, results in a pellet fraction X; and a centrate Cᵢ, wherein the centrate Cₓ of centrifugation step x, wherein x is an integer ranging from 1 to n−1, is subjected to centrifugation in centrifugation step x+1,
wherein the centrifugation steps are performed using disc stack centrifuges wherein the feed enters under pressure through a nozzle at the bottom of the centrifuge in the liquid phase already present in the centrifuge and is accelerated to rotor speed, wherein the centrate Cₙ obtained in centrifugation step n has a wet solids content of 0.5 wt. % or less, based on the total weight of the centrate Cₙ, and
wherein any pellet fraction X; is optionally subjected to mechanical pressing resulting in a pressed pellet fraction Xᵢ′ and an aqueous stream F_{c,i};
e) subjecting centrate Cₙ obtained in centrifugation step n of step (d) to a microfiltration step resulting in a permeate Pₑ and a retentate Rₑ, wherein the retentate Rₑ is optionally subjected to mechanical pressing resulting in a pellet fraction Rₑ′ and an aqueous stream Fₑ;
f) subjecting the permeate Pₑ from microfiltration step (e) to an ultrafiltration step, optionally performed as diafiltration step, resulting in a permeate P_{f} and a retentate R_{f};
g) subjecting the retentate R_{f} obtained in step (f) to hydrophobic column adsorption to provide a column permeate P_{g} and a retentate R_{g} remaining on the static phase of the hydrophobic column; and
h) drying the column permeate P_{g} obtained in step (g) to provide a soluble functional protein isolate and water;
wherein the method further comprises a recycling step comprising:
(AA) recycling at least part B of retentate R_{b}, or at least part Dᵢ of stream Xᵢ, wherein i is an integer selected from 1 to n, or at least part E of retentate Rₑ, or combinations thereof, to the coarse physical separation step in step (b);
(BB) when mechanical pressing is performed in any one of steps (b), (d) or (e), recycling at least part B′ of aqueous stream F_{b}, or at least part Dᵢ′ of aqueous stream F_{c,i}, wherein i is an integer selected from 1 to n, or at least part E′ of aqueous stream Fₑ, or combinations thereof, to the mild treatment step in step (c);
(CC) recycling at least part Dᵢ of stream Xᵢ, wherein i is an integer selected from 1 to n, or at least part E of retentate Rₑ, or combinations thereof, to the first centrifugation step in step (d); or
(DD) when mechanical pressing is performed in step (d) or (e), recycling at least part Dᵢ of stream Xᵢ, or at least part Dᵢ′ of aqueous stream F_{c,i}, wherein i is an integer selected from 1 to n, or at least part E of retentate Rₑ, or at least part E′ of aqueous stream Fₑ, or combinations thereof, to the first centrifugation step in step (d).

2. The method according to claim 1, wherein the plant material comprises, preferably consists of, green leaves.

3. The method according to claim 1, wherein the plant material comprises, preferably consists of, the green leaves of sugar beet, alfalfa, chicory, fodder chicory, phacelia, ryegrass, rye, oat, radish, fodder radish, vetches, carrot leaf, chicory leaf, and combinations thereof.

4. The method according to claim 3, wherein the plant material comprises, preferably consists of, the green leaves of sugar beet.

5. The method according to claim 2, wherein the green leaves comprise more than 5 wt. % of dry matter.

6. The method according to claim 1, wherein the mechanical disruption of step (a) is performed by screw press homogenization, milling, pulsed electric field treatment, crushing, slicing, or combinations thereof.

7. The method according to claim 1, wherein at least one reducing agent is added before, during or after step (a), wherein the at least one reducing agent is preferably selected from bisulfite salts, such as alkali cation bisulfite salts, more particularly sodium bisulfite.

8. The method according to claim 1, wherein an acid or base is added before, during or after step (a), preferably after adding the at least one reducing agent, to establish a pH of the plant juice obtained in the mechanical disruption step of about 6-8.

9. The method according to claim 1, wherein the method is performed under an inert atmosphere, preferably under an atmosphere of molecular nitrogen (N₂).

10. The method according to claim 9, wherein before, during or after step (a), preferably before step (a), the molecular oxygen (02) in the material to be processed is displaced with molecular nitrogen (N₂).

11. The method according to claim 1, wherein the coarse physical separation step (b) is performed using a dewatering press or filter press wherein the plant juice is separated from the pulp comprising disrupted cells by squeezing the mush against a filter element such as a screen, filter or sieve and collecting the plant juice through the filter element, wherein preferably a negative pressure or vacuum is applied to the permeate-side of the filter element.

12. The method according to claim 1, wherein mild treatment step (c) comprises exposing the extracted juice to a temperature of between 20 and 55° C., preferably between 25 and 55° C., more preferably between 30 and 50° C., even more preferably between 40 and 50° C.

13. The method according to claim 1, wherein one or more flocculants chosen from the group consisting of salts comprising divalent cations or trivalent cations are added to the permeate P_{b} obtained in step (b) before or during step (c).

14. The method according to claim 13, wherein one or more flocculants chosen from the group consisting of salts comprising divalent cations or trivalent cations are added to the permeate P_{b} obtained in step (b) before or during step (c) and wherein the temperature during step (c) is between temperature between 20° C. and 55° C., preferably between 20 and 50° C., more preferably between 20 and 40° C. still more preferably between 20 and 30.

15. The method according to claim 1, wherein centrifugation in step (d) is performed between 10000 and 20000 rpm for a period between 30 seconds and 2 minutes.

16. The method according to claim 1, wherein the centrifugation in step (d) does not involve the use of decanter centrifuges, sedicanters or decanters.

17. The method according to claim 1, wherein n is 4.

18. The method according to claim 1, wherein the retentate R_{b} is subjected to mechanical pressing resulting in a concentrated fibre stream R_{b}′ and an aqueous stream F_{b}.

19. The method according to claim 1, wherein one or more pellet fractions Xᵢ, wherein i is an integer between 1 and n, are subjected to mechanical pressing resulting in one or more pressed pellet fractions Xᵢ′ and one or more aqueous streams F_{c,i}.

20. The method according to claim 1, wherein the retentate Rₑ is subjected to mechanical pressing resulting in a pellet fraction Rₑ′ and an aqueous stream Fₑ.
